SEQUENCE LISTING (7 sequences)

SEQ ID NO: 1 - protein, 184 aa, Homo sapiens
MADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 2 - DNA, 552 bp, Homo sapiens
atggccgaccacctgatgctcgccgagggctaccgcctggtgcagaggccgccgtccgccgcggccgcccatggccctcatgcgctccggactctgccgccgtacgcgggcccgggcctggacagtgggctgaggccgcggggggctccgctggggccgccgccgccccgccaacccggggccctggcgtacggggccttcgggccgccgtcctccttccagccctttccggccgtgcctccgccggccgcgggcatcgcgcacctgcagcctgtggcgacgccgtaccccggccgcgcggccgcgccccccaacgctccgggaggccccccgggcccgcagccggcgccaagcgccgcagccccgccgccgcccgcgcacgccctgggcggcatggacgccgaactcatcgacgaggaggcgctgacgtcgctggagctggagctcgggctgcaccgcgtgcgcgagctgcccgagctcttcctgggccagagcgagttcgactgcttctcggacttggggtccgcgccgcccgccggctccgtgagctgc

SEQ ID NO: 3 - DNA, 1248 bp, Homo sapiens
cttcagggaccgcacggtgccgggtctcccgccgcaaagcagccgctggcccgggccggggaagccggcgctgaccaccgcgcgctgtccccgcaggcagccggcctgccgccatggccgaccacctgatgctcgccgagggctaccgcctggtgcagaggccgccgtccgccgcggccgcccatggccctcatgcgctccggactctgccgccgtacgcgggcccgggcctggacagtgggctgaggccgcggggggctccgctggggccgccgccgccccgccaacccggggccctggcgtacggggccttcgggccgccgtcctccttccagccctttccggccgtgcctccgccggccgcgggcatcgcgcacctgcagcctgtggcgacgccgtaccccggccgcgcggccgcgccccccaacgctccgggaggccccccgggcccgcagccggcgccaagcgccgcagccccgccgccgcccgcgcacgccctgggcggcatggacgccgaactcatcgacgaggaggcgctgacgtcgctggagctggagctcgggctgcaccgcgtgcgcgagctgcccgagctcttcctgggccagagcgagttcgactgcttctcggacttggggtccgcgccgcccgccggctccgtgagctgctgagggcggccggcgcccgcccggcgtgccggagaggagaaagggcccgactgcccgccggaccctgcacccagcgactgggccccgcgcgcgccctccgcgagggtggaggcggcggctgtgtgcgcagggcccggcaccggactgggaccctggcgtccctccaggccttgcctcctgcgggaggacagtttggcttcacttctctgaccccagcctcggccgtaaagtgaaagagaccggaccagcttcagctttcggactctggttcttggatcgtgtcctctccccctcgccgccctcttcccccaatctgagccattgcaggcctctgcctgctgccccctctctcctcgggatcgggtccccagagccaccatctcctgagcctcccaccccgctgcctgggccctgtggttgctgggcctcccacctcaaggaggggaaggttgtacagcccgaacccgtggagcaatgccctgtctggcctcaaaaccaaaataaaactgggtcactttacagtcttgccgtttcatttccttatcaccccgagccctgactatatttagcttccaaagtccaa

SEQ ID NO: 4 - DNA, 47 bp, Artificial Sequence
  Description of Artificial Sequence Primer TS02.2
ggccgggatccaccatggccgaccacctgatgctcgccgagggctac

SEQ ID NO: 5 - DNA, 41 bp, Artificial Sequence
  Description of Artificial Sequence Primer TS02
ggccgggatccgccgaccacctgatgctcgccgagggctac

SEQ ID NO: 6 - DNA, 46 bp, Artificial Sequence
  Description of Artificial Sequence Primer TS04
ggccggtcgactcagcagctcacggagccggcgggcggcgcggacc

SEQ ID NO: 7 - DNA, 3970 bp, Homo sapiens
ctagaaacaaatgaggtctgaccatagcaacactttcagaacaaaggccagatcatgtcactcctctgccaaagtccttgcaaggcctcaatgcagttggcctccctgctaccctcctagccccactgcactctgtccacactcccttagggctgcggctcacccttgcctaaggcttccccctaactcagaggtccttaatcagaatcacccgcagggcttattaaaacacagatgctgaattccagcccagtttctgattcagaaactgatttgagtctggtgtacggtcctagaatgtgcatttctaatggggtccctggtaaggctgacacttcaggacttgttgctacagttttagaagtgctgctccagcgttctctctgctgatacggctttttttttttttcttcttttaaagatagagtcttgctttgttgcccaggctggagtgcagtggcaccatctcggctcactgcaacctctgcctcctgggttctggcgattctcctgcctcagcctcctgagtagctgggactacaggtgcgtgccaccacacccagctaatttttgtactttttgtagagacggggtttcaccatgttggccaggctgatatcaaattcctgacctcaagtgacctacccacctcagcctcccaaagtgctgggattacaggcgtaagccaccacgcccagcccgcttttgccccagataccccacttaatactacaacttgctcccctcctcccctgcaccccaacctccttcctactctatttttccttttttttttttgtcttttctctttttttttttttttttttgagatggagtctcactctgtcgctggggctggagtgcagtggcgctatctaggctcactgcaacctccgcctcctgggttcaagcaattctcctgcctcagcctcttgagtagctgagattacaggcgtgtgctaccacacccagctaatttttgtatttttagtcgagacagggtttcaccatgttggccaggctggtctcaaactcctatatcaggtgatctgcctaccttggcctcccaaagtgctgggattacaggtgtgagccaccatgcccggcctaaagaacttcttaaacattagttacacacacatttttttttaaatggaaaatcatctggtggtgcaatggctcccaaatgctggtgacctaagaccaatgatggcagaataattttttgtttgttttgtttgtttttttttgttttttgagagagggtctccctacatcacccgggctggagtgcagtggtgtgatcatagctcactgtagcctcaacttcccaggttcaagtgatcctcccacctcaacctcccaagtagcaaggactacaagtgcacaccaccatgccaagttaattttttattttttcagaaacaggggtctcaaactcctgagctcaagggatctacttacctcggcctcccaaagtgctgggattacaagcgtgagccactcacctggcctgcttgttgatttttttgagacaagatctctgtcgcccaggctggagtgcagtggtgccatcatggctcactgcagcctccacctccagggctcagctaatcctcccacctgagcctcctgggtagctcagactacagatacatgccactatgcccaactaattatttaactttttgtagagacacggtctcactatgtttcccaggctggtcttgagctcctgggctcaagcaatcctctcactttcatctcccaaattgggcagattttccgtctttatcaaagcccccactcacccaccccttaccccccacatctccagataagtgtgaggcatggccaggtttaagaatttctgtgagaagccttacctgtagagaaagattcaggagaataaattccagggtgtagctttttacactttgcaaaaatgtgaccacacccatggtcatttatcactacccacaagctctcaggctgaccaggcaagatcaccgtctccctttcacagagcagaaagctgaggccctggggaatcaagtaatactccccgctcaacactcaacagtcacagctggttggtggtggaggtcacacacctgtgggtggcagagatatgattagaacttgggcctctggctgggcgcagtggctcatgcctgtaatcctagcactttggaagcctgaggtgagagggtcacttgaggccagggattcgagataagcctgaggccctcctcactcccccacgccgcccctcccccccgctcccttctctatttaaaaaaataagaagaaagagggaagaaaagaccttgggcccctgaattctcactctagggctctcccctctacatcaactggataaacccaagttccaggaagtcccaaggatgccacaggtcgggggaaggtcagagaagggagagagagtgtcaaatgcgtggagggtaaggttctgggaggctgtctcctggccacaggaaagactccagttgccctaaggacttggaaaacacatggcattcacacgctaactctggctgcctgtgacctcaggacagccactttctctttgtgtgcttctccttatcactgaaatggggctgataatccatgccctgcccccaacaaaatacttgtagaatgtaaagcactttaaaagtccaagttattattgctgttatcaagagaaggccctttgggacagaacttaaatagccttggccgggcgcggtggctcacgcctgtaattccagcactttgggaggccgaggcgggcggatcacgaggtcagcagatcgagaccatcctggctaacacggtgaaaccccgtctctactaaaaatacaaaaaaaattagccgggcgtggtagcgggcgcctgtagtcccagctactcgggagactgaggcaggagaatggcgtgaacccgggaggcggagcttgcagtgagccaagacagcgccactgcagtccagcctgggcgaaagagcgagactccgtctcaaaaaaaaaaaaaaaaagaacttaaatagcctttctttcatttaagggacactttttgagcatctaccacataccaggcactgtgctaggcactgtgcacacatgccctcacttacccctcacaaagggtcagtgctgctgttctttaccttttacagatgaagaaattgaggctcagctgagagttgcaattcattccatcattagttcattcactcagcttttactaaaagcctgctctgggccaggctctgggccaagtgctgagaagaagaagatgaacaagtccttcaggagacgtgcccataaattaataacaactaagtacatgcttcaacagacaaggtgcagtgggaactagagggggagcaacaggctctcctgggggttatgggaatggacagctgacctcctgcggggaccctgcaccgctaaggtgcatgcgccctgtgtagctgtgcagtctcactttgatcaaggctttgctgtgtatttccaggaagcctcttccgccactgagcctcggtctctacctggaaagttaaaaggttgcgtaggcctgacatcctgcagctcttggggggcctaaaactctggccctcccaccccaccttctggtcagttcaagctctaccccagccaagtccgattccgaagccctgagggccagaccgaatccctccgaaagtgccaaataccccgccccagggcggtgctcagagcctggggcgtggccggagtcccaaggggcggggttccagtgggaggggcggggccaagacctagatgcaggcgtgcgcggcccgcccagaagcgtctcgcccagccaatgagcgtccgagggcggggaagccccgcctctgggtataagaatacgccgagcccagctacgcgacgcggaggtttcggagcactacaggttgcgggcctttgtgaccccaggctgc

(19) United States
(12) Patent Application Publication
(10) Pub. No.: US 2004/0053231 A1
(43) Pub. Date: Mar. 18, 2004

(54) Transcription transactivator protein

(76) Inventors: Shoumo Bhattacharya, Lonsdale Road Oxford (GB); Jose Braganca, Oxford (GB); Tracey Swingler, Oxford (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/221,926

(22) PCT Filed: Mar. 16, 2001
(86) PCT No.: PCT/GB01/01201

(30) Foreign Application Priority Data
Mar. 17, 2000 (GB) 0006572.2

(51) Int. Cl. C12Q 1/68; C07H 21/04; C12N 9/06

(52) U.S. Cl. 435/6; 435/69.1; 435/191; 435/320.1; 435/325; 536/23.2

(57) ABSTRACT

Novel transcription transactivator protein of the CITED family, designated HCITEDX. Nucleic acids encoding the protein and uses of the protein are also provided.

### FIELD OF THE INVENTION

The present invention is concerned with a novel transcription transactivator protein which is a member of the CITED family. This protein, designated HCITEDX, has potential activity in the control of hypoxia signalling and in the activation of genes involved in cholesterol uptake, cholesterol biosynthesis and in the control of inflammation.

### BACKGROUND OF THE INVENTION

The cellular response to hypoxia is of fundamental importance in the pathophysiology of ischaemic heart disease, stroke and tumour vascularization. It is mediated by the transcription factor HIF-1 (hypoxia-induced factor-1), which consists of HIF-1α and ARNT proteins (reviewed in Ratcliffe, 1998; Semenza, 1997). HIF-1α is an unstable protein. Hypoxia induces HIF-1α stabilisation (by blocking its degradation by a VHL-containing complex), nuclear localisation, heterodimerisation with ARNT, DNA-binding and recruitment-of the nuclear proteins p300/CBP (Kallio, 1998; Bhattacharya, 1999; Maxwell, 1999). This results in the transcription of several hypoxia-induced genes, e.g. erythropoietin, vascular endothelial growth factor and inducible nitric oxide synthase, which play important roles in the cellular defence against hypoxia.

P300 and its paralog CBP (CREB-binding protein) are ubiquitously expressed nuclear proteins. They link several signal-activated DNA-bound transcription factors to the transcription machinery, which includes RNA polymerase II, and chromatin modifying activities such as histone acetyltransferases. Mutations in CBP result in Rubenstein-Taybi syndrome (Petrij, 1995), a disease characterised by cranio-facial anomalies, mental retardation and a high (20%) incidence of congenital cardiac defects (Pyeritz, 1997). It is likely that p300 and CBP function to integrate the multiple signalling inputs that impinge on a cell into a coherent transcriptional output. P300/CBP thus play a critical role in cellular function (reviewed by Shikama, 1997) and in development (Yao, 1998). The present inventors, and others, have established that p300/CBP play a major role in the cellular responses to viral infection via the interferon-α-STAT2 pathway (Bhattacharya, 1996; Paulson, 1999; Hottiger, 1998), and to hypoxia, via the HIF-1 pathway (Arany, 1996; Kallio, 1998; Ebert, 1998; Bhattacharya, 1999).

The present inventors identified a ubiquitously expressed p300/CBP binding protein, called p35srj (Bhattacharya, 1999), which is an alternatively spliced isoform of Mrg1 (Shioda, 1996; Dunwoodie, 1998; Leung, 1999). Both p35srj and HIF-1α bind the CH1 region of p300/CBP. p35srj inhibits the binding of HIF-1α to p300/CBP, and blocks hypoxia-driven transcription (Bhattacharya, 1999). p35srj is itself induced by hypoxia in certain cells.

P35srj binds to p300 through amino acid residues located in the p35srj C-terminus. These residues are conserved in the C-termini of certain other proteins, which have been designated as the “CITED” family (for CBP/p300 Interacting Transactivators with ED-rich tails). Members of this family include Msg1 (CITED1) (Shioda, 1996; Dunwoodie, 1998) and p35srj (CITED2)(Bhattacharya, 1999).

### DESCRIPTION OF THE INVENTION

The present inventors have identified a novel human p300-CH1 interacting protein belonging to the CITED family which has been designated HCITEDX and have isolated a nucleic acid molecule-which encodes this protein. Like its paralog p35srj, HCITEDX binds to p300 via the CH1 domain and inhibits transactivation by HIF-1α. Experimental evidence indicates that this protein functions in the control of hypoxia signalling and the control of genes involved in cholesterol uptake, cholesterol biosynthesis, and in the control of inflammation.

Therefore, in accordance with a first aspect of the invention there is provided a nucleic acid molecule encoding an HCITEDX protein, said protein comprising the amino acid sequence illustrated in SEQ ID NO: 1.

In a preferred embodiment, the nucleic acid molecule comprises the complete nucleotide sequence illustrated in SEQ ID NO: 2.

The terms “nucleic acid” or “nucleic acid molecule” includes single or double stranded RNA, single or double stranded DNA (encompassing both genomic DNA or cDNA and also recombinant DNA molecules), synthetic forms and mixed polymers, both sense and antisense strands. Furthermore, the nucleic acid molecule of the invention may be chemically or biochemically modified or may contain non-natural or derivatized nucleotide bases as will be readily appreciated by those skilled in the art. Possible modifications include, for example, the addition of isotopic or non-isotopic labels, substitution of one or more of the naturally occurring nucleotide bases with an analog, internucleotide modifications such as uncharged linkages (e.g. methyl phosphonates, phosphoamidates, carbamates, etc.) or charged linkages (e.g. phosphorothioates, phosphorodithioates, etc.). Also included are synthetic molecules that mimic polynucleotides in their ability to bind to a designated sequence, for example via hydrogen bonding. Such molecules are known in the art and include, for example, so-called peptide nucleic acids (PNAs) in which peptide linkages substitute for phosphate linkages in the backbone of the molecule.

Also within the scope of the invention are variants of a defined nucleic acid molecule, particularly variants which exhibit only minor base variations, including base substitutions which result in a synonymous codon (a different codon specifying the same amino acid residue) due to the degeneracy of the genetic code. Also encompassed by the invention are naturally occurring allelic variants of the HCITEDX genomic sequence defined herein, in particular allelic variants which exhibit one or more single nucleotide polymorphisms (SNPs).

Libraries of human chromosomal or cDNA fragments may be screened as sources of the nucleic acids of the present invention. Alternatively, nucleic acid sequences according to the invention may be produced using recombinant or synthetic means, for example by PCR amplification of sequences resident in chromosomal DNA or cloned fragments thereof or by RT-PCR amplification starting from total or poly A+ RNA. Liver tissue is a particularly good source of RNA as HCITEDX is highly expressed in this tissue. Generally such techniques are well known in the art (see Sambrook et al. (1989), Molecular Cloning: A Laboratory Manual, Cold Spring Harbor Laboratory Press; F. M. Ausubel et al. (eds.), Current Protocols in Molecular Biology, John Wiley & Sons, Inc. (1994)).

Also provided by the invention are nucleic acid molecules which are capable of hybridising to nucleic acid molecules according to the invention under conditions of high stringency. A nucleic acid molecule is “capable of hybridising” to another nucleic acid molecule, such as a fragment of DNA or an RNA, when a single stranded form of the nucleic acid molecule can anneal to the other nucleic acid molecule under the appropriate conditions of temperature and ionic strength, which conditions would be well known to those skilled in the art (See Sambrook et al. or Ausubel et al., supra).

The term “stringency” refers to the hybridisation conditions wherein a single-stranded nucleic acid joins with a complementary strand when the purine or pyrimidine bases therein pair with their corresponding base by hydrogen bonding. High stringency conditions favour homologous base pairing (i.e. Watson-Crick base pairing) whereas low stringency conditions favour non-homologous base pairing. “High stringency” conditions comprise, for example, a temperature of about 42° C. or less, a formamide concentration of less than about 20%, and a low salt (SSC) concentration (20×SSC stock solution contains 3M sodium chloride, 0.3M sodium citrate, pH 7.0); or, alternatively, a temperature of about 65° C., or less, and a low salt (SSPE) concentration (1×SSPE solution contains 180 mM NaCl, 10 mM NaH₂PO4 and 1 mM EDTA, pH 7.4). For example, high stringency conditions comprise hybridization in 0.5 M NaHPO₄, 7% sodium dodecyl sulfate (SDS), 1 mM EDTA at 65° C. (Ausubel, F. M. et al. Current Protocols in Molecular Biology, Vol. I, 1989; Green Inc. New York, at 2.10.3). “Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1M NaCl.

The nucleic acid capable of hybridising to a nucleic acid according to the invention under high stringency conditions will generally share at least 80%, preferably at least 90% and more preferably at least 95% nucleic acid sequence identity with the nucleic acid molecule according to the invention. Nucleic acid sequence identity (%) is calculated on the basis of an optimal alignment of the sequences to be compared, taking into account insertions or deletions. Optimal sequence alignments may be assembled using one of the computer algorithms known in the art, for example the BLAST program (accessible via www.ncbi.nlm.nih.gov). Most preferably, the nucleic acid capable of hybridising to the nucleic acid molecule of the invention under conditions of high stringency will also encode an HCITEDX protein of the invention, as defined below.

Also within the scope of the invention are nucleic acid molecules encoding CITED family proteins which comprise an amino acid sequence having at least 78% amino acid sequence identity with and a length approximately equal to the amino acid sequence illustrated in SEQ ID NO: 1. Amino acid sequence identity is also to be calculated on the basis of an optimal alignment, taking account of insertions or deletions, such as may be assembled using the BLAST suite of programs.

The nucleic acid molecules of the invention may advantageously be incorporated into expression vectors to allow for expression of the HCITEDX protein of the invention.

As will be readily appreciated by the skilled artisan, the expression vectors will include not only nucleic acid encoding an HCITEDX protein according to the invention but also regulatory sequences operably linked to said nucleic acid, such as promoter regions that are capable of effecting expression of said DNA fragments. The term “operably linked” refers to a juxtaposition wherein the components described are in a relationship permitting them to function in their intended manner.

Regulatory sequences required to effect gene expression generally include promoter sequences to position RNA polymerase at the transcription start site and to direct an appropriate frequency of transcription initiation at this site and also translation initiation sequences for ribosome binding. As would be readily understood by one skilled in the art, the precise nature of the regulatory sequences required to effect expression of the HCITEDX protein will vary according to the nature of the host cell. For expression in a prokaryotic host cell (e.g. the bacterium E. coli) the expression vector would include a promoter, such as the lac promoter, to drive transcription and for translation initiation the Shine-Dalgarno and a translation initiation codon (usually AUG). For expression in eukaryotic host cells, the expression vector may include a heterologous or homologous promoter region, preferably one which is recognised by RNA polymerase II, and optionally one or more additional transcriptional regulatory elements (e.g. enhancer elements), also a terminator sequence and downstream polyadenylation signal, a start codon (usually AUG) and a termination codon for detachment of the ribosome. Such vectors may be obtained commercially or may be assembled from the elements described by methods well known in the art.

Examples of expression vectors according to the invention are plasmids, viral or phage vectors. Such vectors will normally possess one or more selectable markers, such as a gene for antibiotic resistance. Plasmid vectors, including those designed for expression in mammalian cells, generally contain a bacterial origin of replication to allow replication in bacterial host cells.

A nucleic acid molecule according to the invention may be inserted into the vectors described in an antisense orientation in order to provide for the production of antisense RNA. Antisense RNA or other antisense nucleic acids, including antisense oligonucleotides, may also be produced by synthetic means.

The expression vector of the invention may further be adapted for expression of the HCITEDX protein of the invention as an in-frame fusion protein or for the addition of an epitope tag.

An expression vector according to the invention can be used to express the protein encoded therefrom in a suitable host cell or organism. Thus, in a further aspect, the invention provides a process for preparing an HCITEDX protein according to the invention which comprises cultivating a host cell, comprising an expression vector as described above under conditions to provide for expression of a coding sequence in the vector encoding the HCITEDX protein, and recovering the expressed HCITEDX protein. Procedures for incorporation of a cloned DNA into a suitable expression vector, introduction of the expression vector into a host cell, selection of transformed cells harboring the vector, culture of the host cells and recovery of the expressed protein are well known to those skilled in the art, as provided by Sambrook et al. (1989), Molecular Cloning: A Laboratory Manual, Cold Spring Harbor Laboratory Press or F. M. Ausubel et al. (eds.), Current Protocols in Molecular Biology, John Wiley & Sons, Inc. (1994).

In an important aspect, the invention provides an expression vector which is suitable for use in driving expression of the HCITEDX protein in mammalian cells in vivo (or ex vivo). Such expression vectors might be used to provide therapeutic benefit in somatic gene therapy or may be used as research tools to investigate the function of HCITEDX in animal models.

Preferred types of expression vectors for in vivo use are viral vectors, particularly adenovirus-derived vectors, although plasmid expression vectors have also been proposed for use in somatic gene therapy. A number of suitable adenoviral vectors are known in the art. For example, WO 95/00655 describes Ad5 vector systems which are deleted in both the E1 and E3 regions. Another useful adenovirus vector is pCMVAdTrack which is available commercially.

The invention further provides a transgenic cell or non-human transgenic organism comprising a transgene capable of expressing the HCITEDX protein of the invention.

The term “transgene capable of expressing” as used herein means a suitable nucleic acid sequence is which leads to expression of the HCITEDX protein of the invention. Advantageously, the transgene may be present in an expression vector, as described above.

In a second aspect, the invention provides an isolated HCITEDX protein comprising the complete amino acid sequence illustrated in SEQ ID NO: 1.

In a preferred embodiment, the isolated HCITEDX protein is encoded by a nucleic acid molecule according to the first aspect of the invention. Most preferably, the HCITEDX protein of the invention is encoded by a nucleic acid molecule comprising the complete nucleic acid sequence illustrated in SEQ ID NO: 2.

Also encompassed within the scope of the invention are proteins which are substantially homologous to a defined HCITEDX protein but have one or more amino acid changes including conservative substitutions, naturally occurring allelic variants, or in vivo or in vitro chemical or biochemical modifications (e.g. acetylation, carboxylation, phosphorylation, glycosylation etc) which are conservative of biological function. In this context, a “substantially homologous” sequence is regarded as a sequence which has at least 78% or 80%, preferably at least 90% and more preferably at least 95% amino acid sequence identity with the HCITEDX protein of the invention. The “biological function” of the HCITEDX protein is defined herein as the ability to bind to the CH1 domain of p300/CBP and to inhibit transactivation by the transcription factors HIF-1α, EPAS1/HIF-2a or SREBP2 and SREBP1, or NF-κB-p65.

Amino acid changes which are “conservative” are those which permit biological function to be retained although it may be less than or greater than the level of biological function of the wild-type HCITEDX protein. The choice of amino acids for making conservative changes will be well-known to those skilled in the art. The invention also contemplates fragments of the HCITEDX protein which retain equivalent biological function, for example fragments which are deleted for one or more amino acid residues at the N- or C-terminus of the protein, and also variants which contain internal deletions or insertions of one or more amino acid residues but which retain equivalent biological function to the wild type HCITEDX protein.

The invention further provides functional fragments of the HCITEDX protein, the term “functional fragment” referring to an isolated sub-region, domain or fragment of an HCITEDX protein or a sequence of amino acids that, for example, comprises a functionally distinct region of the protein, e.g. the p300/CBP binding domain. In a particular embodiment, the invention provides a polypeptide including a fragment of the carboxy-terminal region of HCITEDX consisting of amino acids 138-170 or amino acids 138-184.

The invention still further provides mutant or variant versions of HCITEDX which contain one or more modifications to the primary amino acid sequence of the protein selected from amino acid substitutions, insertions or deletions. Such modifications may 1) reduce or eliminate an activity of the HCITEDX protein, such as p300/CBP binding; 2) enhance a property of the HCITEDX protein, such as stability in an expression system; or 3) provide a novel activity or property, however it is not intended to limit the invention to mutants or variants which provide such effects.

In particular embodiment, the invention provides a mutant version of HCITEDX which lacks a functional p300 binding domain. In a preferred embodiment this mutant protein is deleted for amino acid residues 138-170.

The HCITEDX protein according to the invention may, for example, be synthesised in a recombinant expression system, synthesised in a cell-free in vitro translation system (e.g. a reticulocyte lysate), chemically synthesised or purified from a tissue or a cell which expresses native HCITEDX.

Also encompassed within the scope of the invention are fusion proteins comprising the HCITEDX protein of the invention and also HCITEDX proteins which are labelled with a protein or polypeptide tag, such as an epitope tag. The HCITEDX protein of the invention may be fused either N-terminally or C-terminally to heterologous protein or peptide fragments including, for example epitope tags which are recognised by a specific antibody to facilitate identification and/or purification of the fusion protein, to the product of a reporter gene (e.g. an autonomously fluorescent protein), or to an enzyme (e.g. Glutathione-S-transferase). In an important embodiment, the HCITEDX protein may be fused to either the DNA binding domain or the activation domain of a bipartite transcriptional activator to create a fusion protein for use in a two-hybrid assay. Fusion proteins will typically be made by recombinant nucleic acid techniques but may be chemically synthesized. A number of vectors especially designed for the expression of recombinant fusion proteins in which an epitope tag or other heterologous polypeptide is added to the N- or C-terminus of a protein of interest are available commercially.

In a further aspect, the invention provides an antibody directed against an epitope of the HCITEDX protein according to the invention. Antibodies to an epitope of HCITEDX can be prepared by techniques which are known in the art. For example, polyclonal antibodies may be prepared by inoculating a host animal, such as a rabbit, with an immunogenic preparation comprising HCITEDX or an immunogenic fragment thereof as the challenging antigen and recovering immune serum. Monoclonal antibodies may be prepared according to known techniques (see, for example ANTIBODIES: A Laboratory Manual, E. Harlow and D. Lane, Cold Spring Harbor Laboratory, Cold Spring Harbor, N.Y.; IMMUNOCHEMISTRY 1 and 2: A practical approach (1997), A. P. Johnstone and M. W. Turner, Eds., IRL Press at Oxford University Press).

The present invention also advantageously provides oligonucleotides comprising between about 10 and 50 consecutive nucleotides of the nucleotide sequence illustrated in SEQ ID NO: 2 or the complement thereof. The oligonucleotide molecules of-the invention are preferably from 10 to 50 nucleotides in length, even more preferably from 15-30 nucleotides in length, and may be DNA, RNA or a synthetic nucleic acid, and may be chemically or biochemically modified or may contain non-natural or derivatized nucleotide bases, as will be readily appreciated by those skilled in the art. Possible modifications include, for example, the addition of isotopic or non-isotopic labels, substitution of one or more of the naturally occurring nucleotide bases with an analog, internucleotide modifications such as uncharged linkages (e.g. methyl phosphonates, phosphoamidates, carbamates, etc.) or charged linkages (e.g. phosphorothioates, phosphorodithioates, etc.). Also included are synthetic molecules that mimic polynucleotides in their ability to bind to a designated sequence to form a stable hybrid. Such molecules are known in the art and include, for example, so-called peptide nucleic acids (PNAs) in which peptide linkages substitute for phosphate linkages in the backbone of the molecule. An oligonucleotide molecule according to the invention may be produced according to techniques well known in the art, such as by chemical synthesis or recombinant means.

The oligonucleotide molecules of the invention may be double stranded or single stranded but are preferably single stranded, in which case they may correspond to the sense strand or the antisense strand of the HCITEDX gene. The oligonucleotides may advantageously be used as probes or as primers to initiate DNA synthesis/DNA amplification. They may also be used in diagnostic kits or the like for detecting the presence of HCITEDX nucleic acid sequences. These tests generally comprise contacting the probe with a sample of test nucleic acid under hybridising conditions and detecting for the presence of any duplex or triplex formation between the probe and complementary nucleic acid in the sample. The probes may be anchored to a solid support to facilitate their use in the detection of nucleic acid sequences according to the invention. Suitable solid supports include DNA chips. Preferably, they are present on an array so that multiple probes can simultaneously hybridize to a single sample of target nucleic acid. The probes can be spotted onto the array or synthesised in situ on the array. (See Lockhart et al., Nature Biotechnology, vol. 14, December 1996 “Expression monitoring by hybridisation to high density oligonucleotide arrays”. A single array can contain more than 100, 500 or even 1,000 different probes in discrete locations.

Most preferably, the oligonucleotide molecules of the invention represent ‘unique’ fragments of the HCITEDX gene, meaning that the sequence is unique in the human genome.

The isolation of HCITEDX and the elucidation of its function also enables the development of a number of therapeutic agents.

The HCITEDX protein itself, or any fragment, variant or synthetic analogue thereof which retains an ability to inhibit transcription transactivation substantially equivalent to that of the natural HCITEDX protein, may be therapeutically useful in a number of different indications, particularly the reduction of cholesterol biosynthesis and inflammation, prevention of tumour angiogenesis and treatment of any other conditions in which a patient would benefit from a reduction in the transcriptional response to hypoxia. Thus, the invention provides for a medicament comprising an HCITEDX protein, or a fragment thereof together with a pharmaceutically acceptable diluent, carrier or excipient.

The medicament of the invention may be administered by any conventional route, including injection or infusion. For intravenous use, the HCITEDX protein may be administered in commonly used intravenous fluid(s), for example physiological saline, Ringer's solution or 5% dextrose, and administered by direct injection or infusion. Other routes of administration of an HCITEDX protein based drug are also contemplated, e.g. transdermal administration, inhalation or even oral delivery.

In order to facilitate the efficient delivery of functional HCITEDX protein to cells in vivo use may be made of the so-called “protein transduction” or “protein therapy” approach (Schwarze et al., In vivo protein transduction: delivery of a biologically active protein into the mouse. SCIENCE. (1999) 285: 1569-72). In this method, the therapeutic protein is linked, preferably by in-frame N-terminal fusion, to a protein transduction domain (PTD) which mediates transduction into mammalian cells in a process which is independent of specific receptors or transporters. The preferred PTD is an 11 amino acid region of the human immunodeficiency virus TAT protein having the amino acid sequence YGRKKRRQRRR (M. Green and P. M. Loewenstein. Cell, 55, 1179, 1988; A. D. Frankel and C. O. Pabo. Ibid., p 1189), although synthetic variants of this sequence having equivalent function, i.e. which mediate similar or enhanced levels of protein transduction compared to the wild-type sequence, may also be used.

The invention therefore provides compositions comprising the HCITEDX protein linked to a protein transduction domain, preferably by in-frame N-terminal fusion, and also medicaments comprising these compositions together with appropriate pharmaceutically acceptable carriers, diluents or excipients. Compositions containing the protein transduction domain-HCITEDX composition suspended in physiological saline or saline plus 10% glycerol are suitable for in vivo use (Schwarze et al, ibid).

The invention further provides for delivery of a therapeutically effective amount of a nucleic acid encoding HCITEDX to cells either in vivo or ex vivo, i.e. somatic gene therapy. Adenoviral vectors, as hereinbefore described, are the preferred delivery systems for HCITEDX gene therapy, although alternative types of viral and non-viral delivery systems known in the art may also be used in accordance with the invention.

The present invention is further directed to inhibiting expression of the HCITEDX protein in vivo by the use of antisense technology. Antisense technology can be used to control gene expression through either triple-helix formation using an antisense DNA (or modified versions thereof) or inhibition of expression using an antisense RNA, both of which methods are based on binding of a polynucleotide to DNA or RNA. For example, an antisense RNA oligonucleotide, preferably from 10 to 40 base pairs in length, may be designed to be complementary to a portion of the coding region of HCITEDX, most preferably a region corresponding to the N-terminal part of the protein. The antisense RNA oligonucleotide hybridises to the mRNA in vivo and blocks translation of an mRNA molecule into the Akt-3 (antisense—Okano, J. Neurochem., 56:560 (1991); Oligodeoxynucleotides as Antisense Inhibitors of Gene Expression, CRC Press, Boca Raton, Fla. (1998)). A DNA oligonucleotide may be designed to be complementary to a region of the gene involved in the initiation or regulation of transcription (triple-helix—see Lee et al. Nucl. Acids Res., 6:3073 (1979); Cooney et al., Science, 241:456 (1988); and Dervan et al., Science, 251: 1360 (1991), thereby preventing transcription of HCITEDX mRNA.

A pharmaceutical composition in accordance with this aspect of the invention may include a therapeutically effective amount of the antisense nucleic acid in combination with any standard physiologically and/or pharmaceutically acceptable carriers known in the art. “Pharmaceutically acceptable” means a non-toxic material which does not interfere with the activity of the pharmaceutically active ingredients in the composition.

“Physiologically acceptable” refers to a non-toxic material that is compatible with a biological system such as a cell, tissue or organism. Physiologically and pharmaceutically acceptable carriers may include diluents, fillers, salts, buffers, stabilizers, solubilizers etc.

Alternatively, the antisense nucleic acids described above can be delivered to cells by procedures known in the art such that the anti-sense RNA or DNA may be expressed in vivo to inhibit production of HCITEDX in the manner described above.

Antisense compositions according to the invention would be used to reduce or inhibit the expression of HCITEDX in vivo and therefore block the HCITEDX-p300/CBP interaction. This could be of therapeutic benefit in ischaemic heart disease and other conditions which benefit from enhanced angiogenesis.

The pharmaceutical preparations of the invention are to be administered in pharmaceutically acceptable amounts, an effective amount being an amount of a pharmaceutical preparation that alone, or together with further doses, produces the desired response in the condition being treated. The precise amount of the composition administered will, however, generally be determined by a medical practitioner, based on the circumstances pertaining to the disorder to be treated, such as the severity of the symptoms, the composition to be administered, the age, weight, and response of the individual patient and the chosen route of administration.

The present invention further provides for use of the HCITEDX protein of the invention in screening methods for the identification of compounds which interfere with the function of HCITEDX as a transcriptional regulator and may therefore have potential pharmacological activity in the modulation of hypoxia signalling, tumour angiogenesis or cholesterol synthesis.

The present inventors have determined that HCITEDX strongly inhibits transactivation by the transcription factor HIF-1α involved in hypoxia signalling and its paralog HIF-2α and also the sterol-response element binding protein SREBP2, and its paralog SREBP1, and also NF-κE-p65. The inhibitory functions of HCITEDX require binding to the CH1 domain of p300/CBP, as a HCITEDX mutant lacking the p300 binding domain does not have these inhibitory properties. Furthermore, it has been shown by experiment that the carboxy-terminal region of HCITEDX, extending between amino acids 138-184, is required for binding to the CH1 domain of p300. Thus, the screening methods provided by the invention generally involve assays for compounds which modulate the interaction between HCITEDX and the CH1 domain of p300/CBP, for example by directly interfering with the binding of HCITEDX to p300/CBP and/or by interfering with the cytoplasmic sequestration of HCITEDX.

A wide variety of different assay methodologies may be used in accordance with the invention, including labelled in vitro protein-protein binding assays and also cell-based assays, such as two- and three-hybrid screens. Cell based assays based on a two-hybrid approach are particularly preferred. Accordingly, the invention provides a method for identifying compounds which modulate the interaction between HCITEDX and the CH1 domain of p300/CBP, which method comprises:

providing a host cell containing a DNA construct comprising a reporter gene or a counter-selectable marker gene operably linked to a promoter regulated by a transcription factor having a DNA binding domain and an activating domain;

expressing in said host cell a first hybrid DNA sequence encoding a first hybrid protein comprising an HCITEDX protein according to the invention or a fragment thereof including the p300 binding domain fused in-frame to either the DNA binding domain or the activating domain of the said transcription factor;

expressing in said host cell a second hybrid DNA sequence encoding a second hybrid protein comprising p300, CBP or a fragment thereof including the CH1 domain fused in-frame to either the DNA binding domain or the activating domain of the said transcription factor, such that when the first fusion protein comprises the activation domain of the said transcription factor the second fusion protein comprises the DNA binding domain of the said transcription factor and when the first fusion protein comprises the DNA binding domain of the transcription factor the second fusion protein comprises the activation domain;

contacting the host cell with a sample of a candidate compound; and

detecting any binding of the HCITEDX protein or fragment thereof to the CH1 domain of p300/CBP by either detecting the production of any reporter gene product in the said host cell or by applying positive selection for loss of expression of the counter-selectable marker gene.

This cell-based screening method is based upon ‘classical’ two-hybrid methodology (first described in yeast cells by Chien et al., Proc. Natl. Acad. Sci. USA., 88, 9578-9582, 1991). A typical screen might be based on the use of a lacZ reporter gene under the control of the gal4 promoter, the read-out of the screen being β-galactosidase activity which can be easily measured using an appropriate fluorescent or luminescent substrate.

Although the two-hybrid method was originally devised in yeast, the method of the invention may, advantageously, be carried out in mammalian cells. Preferred cell types are hepatocytes or immortalised cell lines derived from hepatocytes, for example the cell line Hep3B (ATCC#HB-8064).

A preferred configuration, exemplified herein, uses the CH1 domain of p300 fused to the DNA binding domain of GAL4 and HCITEDX (or fragment thereof) fused to the activation domain of VP16. The assay read-out is provided by a construct of the GAL4 promoter operably linked to a luciferase gene.

The method of the invention is preferably performed in vitro and can readily be adapted to be performed in a mid-to-high throughput screening format in multi-well microtitre assay plates. Host cells transfected with the two fusion constructs are incubated with a candidate compound and the effect of the compound on the read-out of the assay (e.g. reporter gene expression) is recorded. Compounds which increase the read-out would be scored as enhancing the HCITEDX-p300/CBP interaction, for example by increasing the rate of translocation of HCITEDX from cytoplasm to nucleus (discussed below) or by increasing its binding to p300/CBP. Compounds which decrease the read-out would be scored as reducing the HCITEDX-p300/CBP interaction, either by preventing binding or by disrupting existing binding. Compounds which cause increased binding of HCITEDX to p300/CBP may be useful for inhibiting hypoxia-driven transcription and tumour angiogenesis, and also SREBP2 driven transcription and cholesterol biosynthesis.

In addition to the ‘classical’ two-hybrid approach, other two-hybrid systems have been developed which are more suitable for use in screening for dissociation events and it is within the scope of the invention to make use any of these systems to screen for compounds which affect the HCITEDX-p300/CBP interaction. These systems, generally designated reverse hybrid screens, make use of host cells in which the expression of interacting hybrid proteins increases the expression of a counter-selectable marker that is toxic under particular conditions. Under these conditions, dissociation of an interaction provides a selective advantage, thereby facilitating detection: For example, a few growing yeast colonies in which hybrids fail to interact can be identified among millions of non-growing colonies expressing interacting proteins.

Several reverse hybrid systems known in the art could be used to perform screens in accordance with this aspect of the invention. The first reverse two-hybrid system utilizes a yeast strain, which is resistant to cycloheximide due to the presence of a mutant CYH2 gene. This strain also contains the wild-type CYH2 allele under the transcriptional control of the GAL1 promoter. Expression of the wild-type GAL4 protein is sufficient to restore growth sensitivity to cycloheximide. Growth sensitivity towards cycloheximide is also restored by the co-expression of the avian c-Rel protein and its IκB-α counterpart, p40, as GAL4 fusion proteins. Restoration of growth sensitivity towards cycloheximide requires the association of c-REL and p40 at the GAL1 promoter and correlates with the ability of the c-REL/p40 interaction to activate expression from the GAL1 promoter (Leanna and Hannink, 1996, NAR 24:3341-3347)

Another reverse hybrid system makes use of the most widely used counter-selectable marker in yeast genetics, URA3, which encodes orotidine-5′-phosphate decarboxylase, an enzyme required for the biosynthesis of uracil. Yeast cells that contain wild-type URA3, either on a plasmid or integrated in the genome, grow on media lacking uracil (URA3⁺ phenotype). However, the ura3-encoded decarboxylase can also catalyze the conversion of a non-toxic analogue, 5-fluorooritic acid (FOA) into a toxic product, 5-fluoroacil (Boeke et al., 1984, Mol. Gen. Genet. 197:345-346). Hence mutations that prevent an interaction can be selected from large libraries or random alleles. Similarly, molecules that dissociate or prevent an interaction could be selected from large libraries of peptides or compounds (Vidal et al., 1996, PNAS 93:10315-10320; Vidal et al., 1996, PNAS 93:10321-10326).

A third reversed yeast two-hybrid is based on the GAL80 gene as relay gene. GAL80 encodes a protein that binds to and masks the activation domain of a transcriptional activator, such as GAL4. The reporter genes, which will provide the transcriptional read-out (HIS3 or LACZ), are dependent upon the functional GAL4 for expression. Only when the level of GAL80 masking protein is reduced by interfering with the two-hybrid interaction will Gal4 function as a transcriptional activator, providing a positive transcriptional read-out for molecules that inhibit the two-hybrid protein-protein interaction. An important feature of this reverse two-hybrid system is that the basal level and the half-time of the relay protein, GAL80, can be fine-tuned to provide maximum sensitivity (Powers and Erickson, 1996, WO95/26400).

The inventors have shown using two-hybrid assays that the carboxy-terminus domain (residues 138-184) is the region required for association with p300. Therefore, in the context of two-hybrid assays a “fragment of HCITEDX including the p300 binding domain” includes, but is not limited to, fragments containing amino acids 138-184.

As an alternative to the cell-based two-hybrid approach, the invention also provides in vitro binding assays which may be used to identify compounds which modulate the HCITEDX-p300/CBP binding interaction. A typical in vitro binding assay involves forming an assay mixture comprising a first protein component comprising the HCITEDX protein, or a fragment thereof including the region responsible for binding to the CH1 domain of p300/CBP, a second protein component comprising p300 or CBP and a candidate compound. The protein components will typically comprise purified recombinant HCITEDX and p300/CBP. The protein components may further be labelled with a label which is directly or indirectly detectable, for example a radioactive label such as ³⁵S, a fluorescent or luminescent label, an enzyme or an epitope tag to facilitate the specific detection of one or other of the protein components. Other components of the assay mixture might include, as appropriate, salts, buffer components etc to facilitate optimum protein-protein binding and to reduce background or non-specific interactions of the reaction components.

Typically, a plurality of assay mixtures are run in parallel, containing varying concentrations of the candidate compound. One of these mixtures may contain zero candidate compound to serve as a negative control. An appropriate positive control would typically also be included.

The assay mixture is incubated under conditions which, in the absence of the candidate compound, would allow binding of HCITEDX to the CH1 domain of p300/CBP. Assay parameters, e.g. order of addition of the reaction components, time and temperature of the incubation, buffer composition of the assay mixture etc, may be readily optimised by routine experimentation, as would be well known to one of ordinary skill in the art. For high-throughput screening purposes, the incubation time should ideally be kept to a minimum.

Following the incubation step, specific binding of HCITEDX to the CH1 domain of p300/CBP is detected by any convenient method. If one or other of the protein components carries a label which is directly or indirectly detectable then specific binding of HCITEDX to p300/CBP can be detected by detecting the presence of the label. Detection of one or other of the protein components may also be accomplished using a specific antibody, e.g. anti-HCITEDX, in an ELISA type assay.

In vitro binding assays may also commonly include a washing or separation step after incubation and prior to detection of specific binding in order to separate bound from unbound reaction components, although for the purposes of high-throughput screening it is advantageous to keep any washing stages to a minimum. A variety of means can be used to effect separation of bound from unbound components. Conveniently, at least one of the protein components may be immobilised on a solid support, for example a microbead, a resin particle or the wells of a microtitre assay plate. Separation may then be effected by, for example, washing the wells of the microtitre plate with a wash solution, washing microbeads or resin particles and then separating them from the bulk solution by centrifugation or, where the support is a magnetic bead, with the use of a magnet.

There are many techniques known in the art which may be used to link protein components to a solid support or matrix. For example purified protein (typically recombinantly synthesised protein) may be simply adsorbed onto the wells of a microtitre plate. Protein components may also be linked to a solid support or matrix via a high affinity specific binding reaction, for example using the binding pairs biotin/avidin, biotin/streptavidin or GST/glutathione. In this case, the protein component may be advantageously-synthesised as a fusion protein containing one component of the binding pair, the other component of the binding pair being linked to the solid support. Expression vectors suitable for use in the synthesis of biotinylated fusion proteins and GST tagged proteins are available commercially (e.g. PinPoint™ system from Promega Corp.; pGEX system from Amersham Pharmacia Biotech).

Preferred in vitro assay configurations are listed below. However, it is to be understood that these are given only by way of example and are not intended to be limiting to the invention:

Assay 1

In this embodiment the HCITEDX protein is radioactively labelled, for example by in vitro translation in a cell-free system incorporating an ³⁵S-labelled amino acid. The p300 protein (or CBP or a CH1 domain fragment of p300 or CBP) is synthesised as a recombinant GST-fusion (i.e., synthesised in E. coli using an expression vector, such as pGEX, adapted for the expression of an in-frame GST fusion protein), purified and immobilised on glutathione agarose beads (SIGMA). The immobilised p300 is then mixed with ³⁵S-labelled HCITEDX in an aqueous solution in the presence of the candidate compound. Specific binding of HCITEDX to the p300 CH1 domain is assayed by SDS-PAGE and autoradiography of bound HCITEDX.

Assay 2

In this embodiment one of the protein components, preferably the p300 protein (or CBP or CH1 domain fragment or p300 or CBP), is linked to a solid support containing a scintillant and the other protein component (HCITEDX) is radiolabelled, for example by in vitro translation with an ³⁵S-labelled amino acid. The two components are mixed in aqueous solution in the presence of the candidate compound. Specific binding of HCITEDX to the p300 CH1 domain is determined by detecting the amount of light emission from the scintillant.

This method is based on the scintillation proximity assay (SPA™) from Amersham, which is widely used in high-throughput screening. SPA beads linked to the first protein component are incubated for 30 minutes to one hour with a sample containing the radioactively labelled second protein component. Upon binding of the two proteins, the radioactivity emitted by the labelled protein is brought into close proximity with the bead containing scintillant and therefore induces light emission from the scintillant. The free labelled protein in the sample (non-bound) will not be held in sufficiently close proximity to the beads to induce light emission. Compounds which disrupt the binding of the interacting proteins will cause a decrease in the amount of light emitted during the experiment.

As would be readily apparent to persons skilled in the art, this assay may be carried out in either orientation, i.e. using HCITEDX linked to the solid support containing scintillant and a radioactively labelled P300 protein or using p300 protein linked to the solid support containing scintillant and a radioactively labelled HCITEDX.

Assay 3

In this assay the first protein component (usually that comprising the HCITEDX protein) is linked to a label which is directly or indirectly detectable. The second protein component (the p300 protein, CBP protein or CH1 domain fragment) is immobilised on a solid support or matrix, such as a microbead or the well of a microtitre plate. The two protein components are mixed in an aqueous solution in the presence of a candidate compound and specific binding of HCITEDX to the p300 CH1 domain is determined by detecting the presence or absence of the label. For this assay format it is preferred to include a washing step following the incubation in order to remove unbound labelled reaction components and excess compound before carrying out the detection step.

Many different types of label known in the art may be used in accordance with this aspect of the invention. The use of epitope tags, such as HA, MYC, FLAG, GST and His tags, which are detectable using a specific antibody is particularly preferred. Alternatively, the assay may use a directly detectable fluorescent label, such as GFP or any of the other autonomously fluorescent proteins known in the art.

Assay 4

A further assay may be based on the use of fluorescence energy transfer (FRET), a technique well known in the art for the detection and quantitative measurement of a whole range of specific binding interactions in biological systems, to screen for compounds which modulate the binding of HCITEDX or a fragment thereof to the CH1 domain of p300/CBP.

The general principles of FRET are as follows: one component of a binding pair is labelled with a first fluorophore (hereinafter referred to as the donor fluorophore) and a second component of the binding pair is labelled with a second fluorophore (hereinafter referred to as the acceptor fluorophore). It is an essential feature of the FRET technique that the fluorescence emission spectrum of the donor fluorophore overlaps with the absorption spectrum of the acceptor fluorophore, such that when the two components of the binding pair bind to each other, bringing the donor and acceptor fluorophores into close proximity, a proportion of the fluorescent signal emitted by the donor fluorophore (following irradiation with incident radiation of a wavelength absorbed by the donor fluorophore) will be absorbed by the proximal acceptor fluorophore (a process known in the art as fluorescence energy transfer) with the result that a proportion of the fluorescent signal emitted by the donor fluorophore is quenched and, in some instances, that the acceptor fluorophore emits fluorescence. Fluorescence energy transfer will only occur when the donor and acceptor fluorophores are brought into close proximity by the specific binding reaction. Thus, in the presence of a compound which disrupts the specific binding, the amount of quenching is reduced resulting in an increase in the intensity of the fluorescent signal emitted by the donor fluorophore or a fall in the intensity of the signal emitted by the acceptor fluorophore).

Suitable pairs of donor and acceptor fluorophores are well known in the art. A preferred combination comprises fluorescein as donor fluorophore and rhodamine as acceptor. Techniques for the conjugation of these fluorophores to proteins and peptides are well known in the art.

FRET-based assays are usually performed in vitro but a similar approach may be used in vivo in a suitable host cell, for example a mammalian cell line. An in vivo assay may be based on the use of genetically encoded donor and acceptor fluorophores which can be expressed as fusion proteins fused in frame to HCITEDX and to p300/CBP. This can be readily accomplished by transforming or transfecting the cell or organism with appropriate expression vectors arranged to express the fusion proteins.

In a preferred embodiment the genetically encoded donor and acceptor proteins may be autonomous fluorescent proteins, for example GFP variants or GFP homologues, which exhibit different fluorescent properties and which have suitably overlapping emission/absorption spectra.

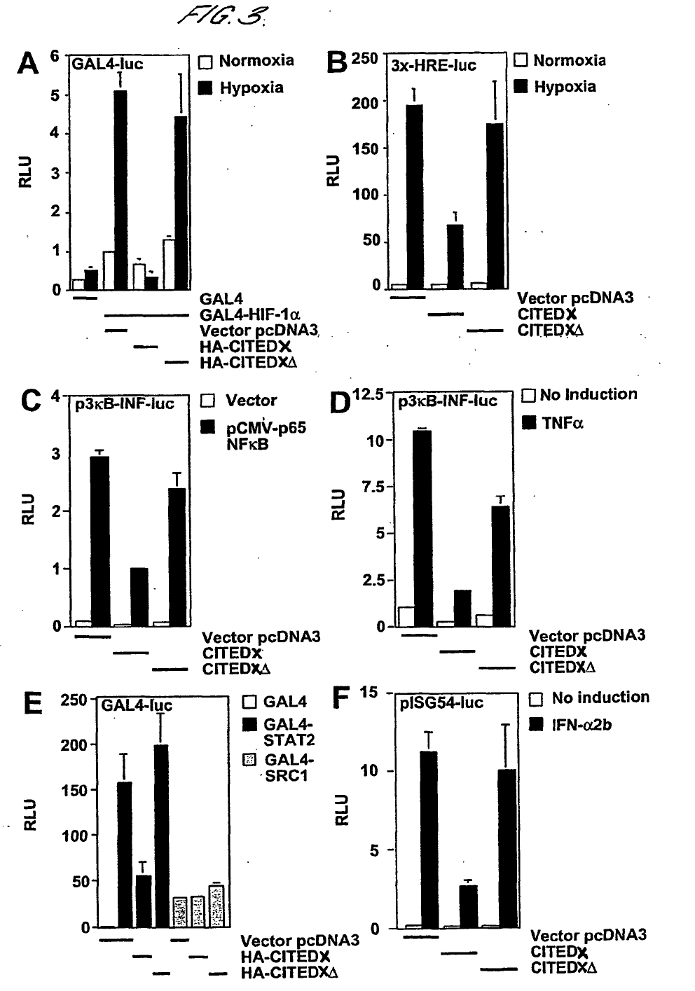

As would be readily appreciated by the skilled artisan, the above-described in vitro screening methods and also the cell-based two-hybrid methods may all be carried out using fragments, variants or mutant forms of p300 or CBP which include the CH1 domain (see FIG. 3A) and using sub-fragments, isolated domains, variant or mutant versions of the HCITEDX protein which retain the ability to bind to the CH1 domain of p300/CBP. It has been shown by experiment that the carboxy-terminal region of HCITEDX extending between amino acids 138-184 is required for binding to the CH1 domain. Hence, the in vitro and cell-based screening assays described herein may be based on the use of HCITEDX fragments including this region and references to the use of “HCITEDX protein” in this context are to be construed accordingly as encompassing such fragments of the HCITEDX protein.

“p300/CBP” refers to a member of the p300/CBP family of transcriptional co-activators (reviewed in Goodman and Smolik, 2000; Shikama et al., 1997). As aforesaid, the assays of the invention may be carried out using allelic or synthetic variants, mutant forms or sub-fragments of p300/CBP which retain the ability to interact with HCITEDX through the CH1 domain. References in this context to p300/CBP are to be construed accordingly.

It will be appreciated that a wide variety of candidate compounds may be tested using the method of the invention to determine whether they are capable of modulating the interaction between HCITEDX and p300/CBP. The compound may be of any chemical formula and may be one of known biological or pharmacological activity, a known compound without such activity or a novel molecule such as might be present in a combinatorial library of compounds.

The invention still further provides compounds which are identifiable as modulators of the interaction between HCITEDX and the CH1 domain of p300/CBP using the above-listed methods of the invention. Compounds which increase the interaction between HCITEDX and the CH1 domain of p300/CBP may have the effect of blocking hypoxia signalling and thus be useful in preventing tumour angiogenesis and may also block cholesterol biosynthesis, and inflammation. Compounds which prevent or disrupt the interaction may enhance angiogenesis, which could be useful in the treatment of ischaemic heart disease. Compounds which are identifiable as having potential pharmacological activity using the methods of the invention may be used as lead compounds in the further development of drugs with pharmaceutical potential or may themselves be formulated into pharmaceutical compositions.

One further cell-based approach to identifying compounds which modulate the interaction between HCITEDX and the CH1 domain of p300/CBP is based on changes in the sub-cellular localisation of the HCITEDX protein. The present inventors have observed that, in contrast to the previously known CITED family proteins which are predominantly nuclear, HCITEDX is also located in the cytoplasm. Thus it seems that, by analogy with many other transcription factors (e.g. SREBP, HIF-1α, STATs, NF-κB etc), the function of HCITEDX as a transcriptional transactivator is regulated by cytoplasmic sequestration. In a classical two-hybrid assay HCITEDX appears to interact weakly with p300 as compared to the HCITED2/p300 interaction, although the in vitro binding affinities of HCITEDX and HCITED2 for p300 are actually similar. This is because in order to interact with p300, cytoplasmic HCITEDX must first be translocated to the nucleus. In spite of this, HCITEDX is a more powerful suppressor of p300/CBP mediated function than CITED2. Observations in breast cancer patients further suggest that the cytoplasmic sequestration of HCITEDX may be important in disease, since in these patients HCITEDX is effectively stuck in the cytoplasm and does not translocate into the nucleus.

Compounds which modulate the translocation of HCITEDX from the cytoplasm to the nucleus, either in the ‘normal’ state or in a disease state, such as in breast cancer, may have potential pharmacological activity for a range of different disease indications, including modulation of hypoxia, tumour angiogenesis, cholesterol synthesis and the treatment of breast cancer. Accordingly, the invention provides a further cell-based screening method for identifying compounds with potentially useful pharmacological activity, which method comprises:

contacting a mammalian cell expressing an HCITEDX protein according to the invention with a candidate compound and detecting any changes in the cellular localisation of the said HCITEDX protein in the presence of the compound.

This method, hereinafter referred to as the cell localisation assay, may be carried out using any cell type which expresses HCITEDX but is most preferably carried out using host cells, such as an immortalised cell line, which have been transfected with an expression construct encoding a recombinant HCITEDX protein. These cells may be stably or transiently transfected, according to-standard procedures known in the art. The host cell may, advantageously, be an immortalised cell line derived from an hepatocyte, such as Hep3B, or a human osteosarcoma cell line such as U-2 OS (ATCC#HTB-96). In one important embodiment, the host cell may be a human breast cancer cell line.

Advantageously, the HCITEDX protein is labelled in order to facilitate localisation of the protein in the cell. Genetically encoded labels which may be added to HCITEDX by expressing an in-frame fusion are particularly preferred. In a preferred embodiment, the genetically encoded label is an epitope tag, in which case changes in the sub-cellular localisation of HCITEDX are detected by immunofluorescence, according to procedures well known in the art, using an antibody specific for the epitope tag. In a further embodiment, the genetically encoded label may be an autonomously fluorescent protein, such as the Aequoria victoria green fluorescent protein or any of the many GFP variants and equivalents known in the art. In this case, changes in the sub-cellular localisation of HCITEDX may be detected directly, for example using fluorescence microscopy at the appropriate wavelength.

The invention also provides compounds which are identifiable as having potential pharmacological activity using the cell localisation assay. Compounds which increase the translocation of HCITEDX into the nucleus may have the effect of blocking hypoxia signalling, preventing tumour angiogenesis, and/or the effect of reducing cholesterol biosynthesis. Compounds which prevent the translocation of HCITEDX into the nucleus may enhance aniogenesis, which may be useful in the treatment of ischaemic heart disease. Compounds which promote the translocation of cytoplasmic HCITEDX in breast cancer cells might be important leads in the development of anti-breast cancer drugs.

For the sake of clarity, it should be mentioned that although the cell-based two-hybrid assay described above has a transcriptional read-out it is in fact capable of identifying compounds which exert their effects via the cytoplasmic sequestration of HCITEDX as well as compounds which directly affect the binding of HCITEDX to p300/CBP. A lead compound identified using the two-hybrid assay might advantageously be tested in one of the in vitro methods in order to determine whether it acts on HCITEDX-p300/CBP binding and/or in the cell localisation assay to determine whether the site of action is cytoplasmic sequestration.

In still further aspects, the invention also provides assays for compounds which modulate the binding of either NF-κB-p65 or SREBP2 and SREBP1 to the CH1 domain of p300/CBP. In particular, the invention provides assays for compounds which prevent, disrupt or inhibit the binding of either NF-κB or SREBP to the CH1 domain of p300/CBP.

The inventors are the first to observe that SREBP1 (as exemplified by its isoform SREBP1a), and its paralog SERBP2, and NF-κB-65 each interact critically with the CH1 domain of p300/CBP. NF-κB plays a major role in inflammatory or immune responses. Compounds which inhibit, prevent or disrupt the interaction of NF-κB with the CH1 domain of p300/CBP may therefore have utility in the treatment of diseases where uncontrolled inflammatory or immune responses are part of the pathophysiology (reviewed in Ghosh et al., 1998). SREBP proteins transcriptionally up-regulate enzymes involved in cholesterol and lipid biosynthesis (reviewed in Brown and Goldstein, 1999). Compounds which inhibit, prevent or disrupt the interaction of SREBP with the CH1 domain of p300/CBP may therefore have utility in the treatment of diseases such as atherosclerosis where hypercholesterolemia plays a major role.

In a preferred embodiment the above assays may be performed using the CH1 domain of p300/CBP in isolation.

Assays for compounds which modulate the binding of either NF-κB-65, SREBP1 or SREBP2 to the CH1 domain of p300/CBP may take the form of in vitro binding assays or two-hybrid assays, analogous to those described above for identifying modulators of HCITEDX binding to p300/CBP.

In an alternative embodiment, assays for identifying potential modulators of the interaction between NF-κB or SREBP and p300/CBP may be performed as reporter gene assays, in which the read-out of the assay is based on transcriptional activation. A typical reporter gene assay will be carried out using a host cell which expresses NF-κB-p65 or SREBP and the CH1 domain of p300/CBP, most preferably the isolated CH1 domain, and which contains a reporter gene construct comprising a promoter which is responsive to NF-κB or SREBP, as appropriate, operably linked to a reporter gene. The host cell is exposed to candidate compounds, and reporter gene expression measured by appropriate means. Compounds which result in a change in the level of reporter gene expression (as compared to suitable control cells, e.g. cells exposed to zero concentration of test compound) are scored as potential modulators of the interaction between NF-κB or SREBP and p300/CBP.

In the context of this aspect of the invention “NF-κB-65” refers to the p65 subunit of NF-_{κ}B and includes the mouse NF-kB-p65 subunit (GenBank NM_{—}009045, Mus musculus avian reticuloendotheliosis viral (v-rel) oncogene homolog A (Rela), mRNA; AUTHORS: Nolan, G. P., Ghosh, S., Liou, H. C., Tempst, P. and Baltimore, D; TITLE: DNA binding and I-kappa-B inhibition of the cloned p65 subunit of NF-kappa-B, a rel-related polypeptide; JOURNAL: Cell 64, 961-969 (1991); MEDLINE 91160060) and the human NF-kB-p65 subunit (Swissprot Q04206; AUTHORS: Ruben, S. M., Dillon, P. J., Schreck, R., Henkel, T., Chen, C. H., Maher, M., Baeuerle, P. A. and Rosen, C. A.; TITLE: Isolation of a rel-related human cDNA that potentially encodes the 65-kD subunit of NF-kappa B; JOURNAL: Science 251 (5000), 1490-1493 (1991)).

“SREBP” refers to both SREBP2 and its paralog SREBP1.

SREBP2: LOCUS HSU02031 4249 bp mRNA PRI 22-OCT-1994

DEFINITION Human sterol regulatory element binding protein-2 mRNA, complete cds.

ACCESSION U02031

AUTHORS Hua, X., Yokoyama, C., Wu, J., Briggs, M. R., Brown, M. S., Goldstein, J. L. and Wang, X.

TITLE SREBP-2, a second basic-helix-loop-helix-leucine zipper protein that stimulates transcription by binding to a sterol regulatory element

JOURNAL Proc. Natl. Acad. Sci. U.S.A. 90 (24), 11603-11607 (1993)

MEDLINE 94089681

In a still further aspect, the invention provides an isolated fragment of the HCITEDX promoter and for assays based on the use of this promoter, or subfragments thereof which retain transcription regulatory activity, to identify compounds potentially capable of modulating expression from the HCITEDX promoter.

Thus the invention provides a HCITEDX promoter fragment having the sequence of nucleotides illustrated in SEQ ID NO: 7. Also contemplated by the invention are fragments of the complete sequence shown in SEQ. ID NO: 7 which retain promoter activity, in particular the ability to direct a tissue-specific pattern of gene expression, most preferably a tissue-specific gene expression pattern substantially identical to the native HCITEDX gene expression pattern, and also fragments which function as enhancer elements. The promoter and/or enhancer activity of fragments of the sequence shown in SEQ ID NO: 7 can be easily tested using reporter gene assays, for example by constructing a deletion series of the complete fragment. Plasmid vectors containing reporter genes for use in testing the promoter and/or enhancer activity of DNA fragments are available commercially (e.g. the pGL2 and pGL3 vector series from Promega Madison, Wis., USA). Promoter elements required for positioning of RNA polymerase and initiation of basal transcription are likely to be found immediately upstream of the transcription initiation site, whereas enhancer elements and elements required for tissue-specific expression might be found far upstream.

In addition to the functional promoter activity studies outlined above, knowledge of the sequence of the HCITEDX promoter sequence is useful for the construction of homologous recombination vectors for in vivo targeting of HCITEDX in the mouse by promoter sequence alterations.

Isolation of the promoter region of the HCITEDX gene allows the development of reporter gene assays to identify compounds which modulate HCITEDX gene expression. Accordingly, the invention provides a method of identifying a compound capable of modulating expression of HCITEDX from its natural promoter, which method comprises:

providing a recombinant host cell containing a reporter gene expression construct comprising the promoter region of the human HCITEDX gene operably linked to a reporter gene;

contacting the host cell with a candidate compound ; and

screening for expression of the reporter gene product.

The above method of the invention can be used to identify compounds which up-regulate the expression of HCITEDX and hence have potential pharmacological activity.

For the purposes of this application, the term “promoter region of the human HCITEDX gene” may refer to the complete sequence shown in SEQ ID NO: 7, a fragment thereof lacking sequences downstream of the transcription initiation site or a transcriptionally active fragment thereof, to the proximal promoter region, i.e. sequences immediately upstream of the HCITEDX transcription start site which are necessary for correctly positioning RNA polymerase and also to the proximal promoter region plus any additional sequence elements which may be involved in regulating HCITEDX gene expression, e.g. upstream enhancer sequences etc. The promoter region of the human HCITEDX gene, as defined above, is positioned to control expression of a reporter gene encoding a protein product which is directly or indirectly detectable. The juxtaposition of the HCITEDX promoter region and a reporter gene may be referred to herein as a ‘reporter gene expression construct’.

Reporter genes which may be used in accordance with the invention include those which encode a fluorescent product, such as green fluorescent protein (GFP) or other autonomous fluorescent proteins of this type or those which encode an enzyme product, such as for example chloramphenicol acetyl transferase (CAT), β-galactosidase and alkaline phosphatase, which is capable of acting on a substrate to produce a detectable product.

Reporter gene assays using reporter gene expression constructs are well known in the art and commonly used in the art to test the promoter activity of a given DNA fragment. They may also be adapted, as in the present invention, to screen for compounds capable of modulating gene expression.

The reporter gene expression construct is preferably incorporated into a replicable expression vector so that it may be conveniently introduced into the eukaryotic host cell. The eukaryotic host cell must be one which contains the appropriate transcription machinery for RNA Polymerase II transcription, and is preferably a cultured mammalian cell. In a preferred embodiment, the host cell is a cell type which is known to express HCITEDX in vivo or is a transformed cell line derived from a cell type known to express HCITEDX in vivo.

An expression vector may be inserted into the host cell in a manner which allows for transient transfection or alternatively may be stably integrated into the genome of the cell (i.e. chromosomal integration). Chromosomal integration is generally preferred for drug screening because the expression constructs will be maintained in the cell and not lost during cell division, also there is no need to separately control for the effects of copy number.

Stable integration of a reporter gene expression construct into the genome of eukaryotic host cell may be achieved using a variety of known techniques. The most simple approach is selection for stable integration following transfection of a host cell with a plasmid vector. Briefly, a plasmid vector comprising a reporter gene expression construct consisting of the HCITEDX promoter region ligated to a promoterless reporter gene cDNA and also a gene encoding a dominant selectable marker, such as neomycin phosphotransferase, is first constructed using standard molecular biology techniques. The plasmid vector is then used to transfect eukaryotic host cells using one of the standard techniques such as, for example, lipofection. Following transfection stable cell lines in which the plasmid DNA has become randomly integrated into the chromosome are selected with growth on appropriate media. For plasmids carrying the neomycin phosphotransferase gene this is achieved using the antibiotic G418. Plasmid vectors suitable for use in the construction of stable cell lines are commercially available (for example the pCI-neo vector from Promega corporation, Madison Wis., USA).

Stable integration into mammalian chromosomes may also be achieved by homologous recombination, a technique which has, been commonly used to achieve stable integration of foreign DNA into embryonic stem cells as a first stage in the construction of transgenic mammals. Stable integration into eukaryotic chromosomes can also be achieved by infection of a host cell with a retroviral vector containing the appropriate reporter gene expression construct.

It will be appreciated that a wide variety of compounds can be tested using the method of the invention to see whether they are capable of up-regulating HCITEDX gene expression and hence have potential pharmacological activity. The compound may be of any chemical formula and may be one of known biological or pharmacological activity, a known compound without such-activity or a novel molecule such as might be present in a combinatorial library of compounds. The method of the invention may be easily adapted for screening in a medium-to-high throughput format.

Compounds which are identified as being capable of up-regulating HCITEDX gene expression should be further tested in order to establish whether the effect on gene expression is HCITEDX-specific or non-specific. This could be achieved using a control cell containing a control reporter gene expression construct with no HCITEDX promoter sequences.

The invention will be further understood with reference to the following non-limiting examples, together with the accompanying Figures in which:

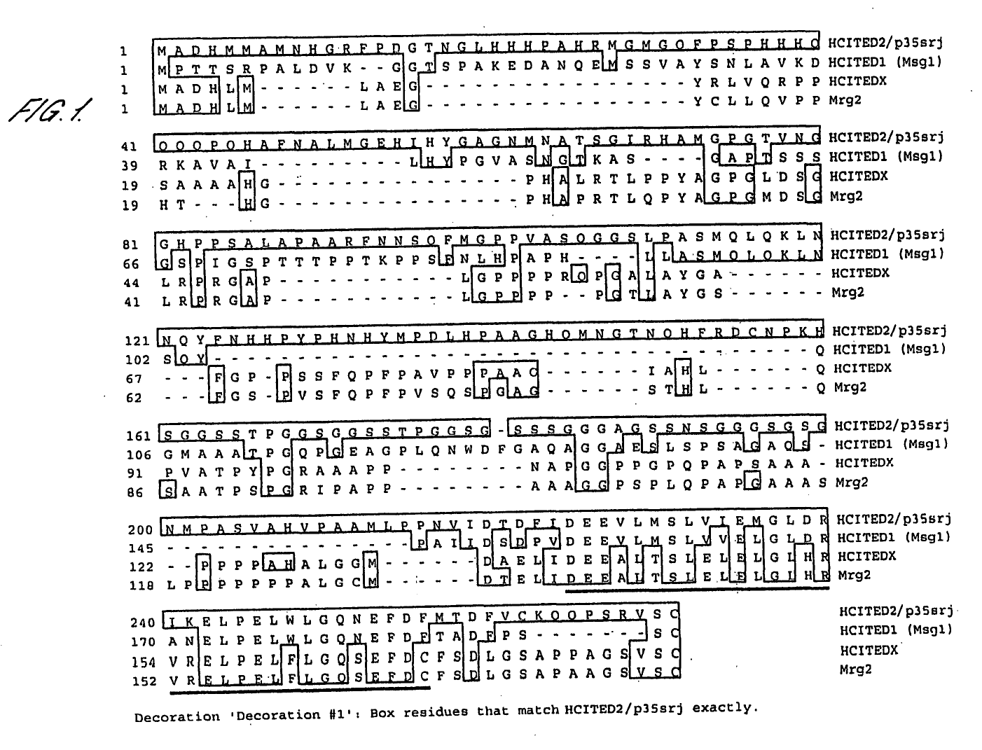

FIG. 1: Clustal alignment of human members of the CITED gene family (HCITED1 (=Msg1), HCITED2 (=p35srj/Mrg1), HCITEDX) and a mouse member of the family, Mrg2. Based on sequence similarity, Mrg2 is likely to be the mouse homologue of HCITEDX. The solid line indicates the 32 amino acids that in HCITED2 are necessary and sufficient for binding p300 CH1 in vitro (Bhattacharya, S. et al. Genes Dev. 1999; 13: 64-75).

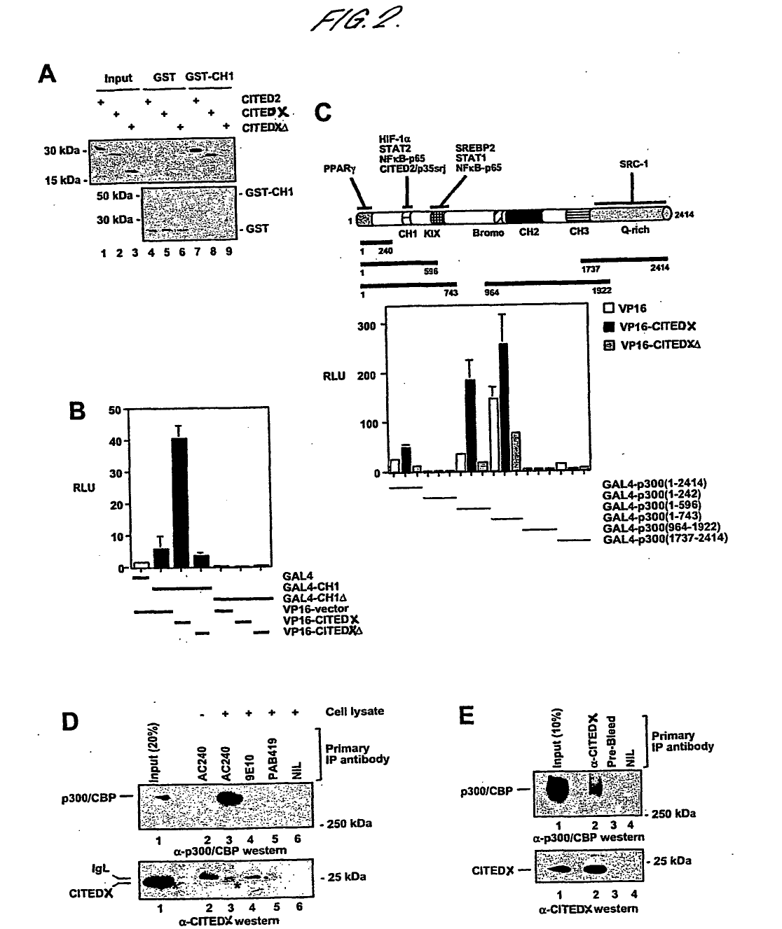

FIG. 2: HCITEDX interacts with p300/CBP. (A) Carboxy-terminal residues of HCITEDX are required for its interaction with the p300-CH1 domain. Top panel: The binding of ³⁵S-labelled CITED2 (lane 1), HCITEDX (lane 2) and HCITEDX mutant lacking the carboxy-terminal residues 138 to 184 (HCITEDXΔ, lane 3) to bacterially expressed GST (lanes 4-6) or GST-p300CH1 (p300 residues 300-528, lanes 7-9) immobilised on glutathione-sepharose beads was tested. Bottom panel: Coomassie stain of the gel showing relative amounts of GST and GST-p300CH1 proteins. (B) HCITEDX interacts with the CH1 domain of p300 in a mammalian two-hybrid system. Hep3B cells were transfected with plasmids expressing the indicated GAL4 and VP16 fusion proteins (40 ng each), 3×GAL4-luc reporter (100 ng) and CMV-lacZ (100 ng). GAL4-CH1 contains the residues 300-528 of p300 fused to the DNA-binding domain of GAL4. GAL4-CH1D lacks residues 346-410 of p300 and served as control. VP16-HCITEDXΔ expresses the VP16 activation domain fused to residues 1-137 of HCITEDX. Results are presented as relative luciferase units (RLU), corrected for lacZ activity, and show the mean±SEM of three independent experiments. (C) The CH1 domain of p300 is necessary for the HCITEDX interaction. Top panel: Schematic diagram of p300 domain structure. P300 and CBP contain multiple conserved regions including three cysteine-histidine rich regions (CH1-3), the KIX domain, a bromodomain and a glutamine-rich region. The regions of p300 used as GAL4-fusions in the mammalian two-hybrid system are indicated below. Numbers indicate the amino acid positions of fragments used. Bottom panel: A mammalian 2-hybrid system was used to assess the interaction of different p300 subregions with HCITEDX. Hep3B cells were co-transfected with the indicated plasmids. Results were analysed as described in B. (D) HCITEDX is present in anti-p300/CBP immunoprecipitates. Top panel: Anti-p300/CBP immunoblot of immunoprecipitates (IP) from ECV304 cell lysates. Immunoprecipitations used anti-p300/CBP monoclonal antibody AC240 (lane 3), and control antibodies (9E10, PAB419, and no primary antibody i.e. rabbit-anti-mouse IgG alone, in lanes 4, 5, and 6 respectively). Lane 1 contains 20% of the extract used for the IP. Lane 2 shows a reaction performed without protein extract. Bottom panel: Anti-HCITEDX immunoblot of above IP reactions. IgL—immunoglobulin light chains. HCITEDX migrates just below IgL, and is-indicated by the asterisk. (E) p300/CBP are present in anti-HCITEDX immunoprecipitates. Top panel: Anti-p300/CBP immunoblot of immunoprecipitates (IP) from ECV304 cell lysates. Immunoprecipitations used anti-HCITEDX polyclonal antibody (lane 2), control pre-bleed serum (lane 3), or no primary antibody (lane 4). Lane 1 contains 10% of the extract used for the IP. Bottom panel: Anti-HCITEDX western blot of above immunoprecipitates.

FIG. 3: HCITEDX inhibits hypoxia, TNF-α and IFN-α activated transcription.

(A) HCITEDX inhibits HIF-1α transactivation. Hep3B cells were transiently co-transfected with GAL4-HIF-1α or GAL4 DNA-binding domain alone (40 ng), 3×GAL4-luc reporter (100 ng), CMV-lacZ (100 ng) and the indicated HA-HCITEDX expressing plasmids (40 ng). HA-HCITEDXΔ plasmid expresses HA-fused to residues 1-137 of HCITEDX. The vector plasmid served as a further control. The effect of HA-HCITEDX proteins on GAL4-HIF-1α transactivation was tested on cells maintained in normoxia (21% O₂), or stimulated with hypoxia (1% O₂) for 16 hours. Results are presented as relative luciferase units (RLU), corrected for lacZ activity, and show the mean±SEM of three independent experiments. (B) HCITEDX blocks the transcriptional activation of a hypoxia response element (HRE). Hep3B cells were co-transfected with 3×HRE-luc reporter (40 ng), CMV-lacZ (100 ng) and the indicated HCITEDX or HCITEDXΔ expressing plasmids, or pcDNA3 vector control (40 ng). The effect of HCITEDX proteins was tested on cells maintained in normoxia, or stimulated with hypoxia for 16 hours. Results are presented as for A. (C) HCITEDX inhibits NF-kB-p65 dependent transactivation. Hep3B cells were transfected with 40 ng of either a plasmid carrying the luciferase gene under the control of wild type NF-kB binding sites (p3×kB-INF-Luc). They were co-transfected with CMV-lacZ (100 ng), pCMV-p65-NF-kB (40 ng), and a plasmid expressing HCITEDX or HCITEDXΔ (40 ng). Results are presented as for A. (D) HCITEDX inhibits TNF-α activated transcription. Hep3B cells were co-transfected with 40 ng of a plasmid carrying the luciferase gene under the control of wild type NF-kB binding sites (p3×kB-INF-Luc), CMV-lacZ (100 ng) and plasmids expressing HCITEDX or HCITEDXΔ (40 ng). Transfected cells were grown for 16 hours in the presence or absence of 10 ng/ml of TNF-α. Results are presented as for A. (E) HCITEDX suppresses transactivation by STAT2 but not by SRC1. The indicated GAL4 proteins (40 ng) were co-transfected with HA-HCITEDX and HA-HCITEDXΔ expressing plasmids or pcDNA3 vector control (40 ng), 3×GAL4-luc reporter (100 ng), CMV-lacZ (100 ng) into Hep3B cells. Results are presented as for A. (F) HCITEDX inhibits IFN-α activated gene transcription. Hep3B cells were co-transfected with 40 ng of a plasmid carrying the luciferase gene under the control of the IFN-α response elements of ISG54 (pISG54-luc), CMV-lacZ (100 ng) and plasmids expressing HCITEDX, or HCITEDXΔ, or the control pcDNA3 vector (200 ng). Transfected cells were grown for 8 hours in the presence or absence of 500 IU/ml of IFN-α2b. Results are presented as for A.

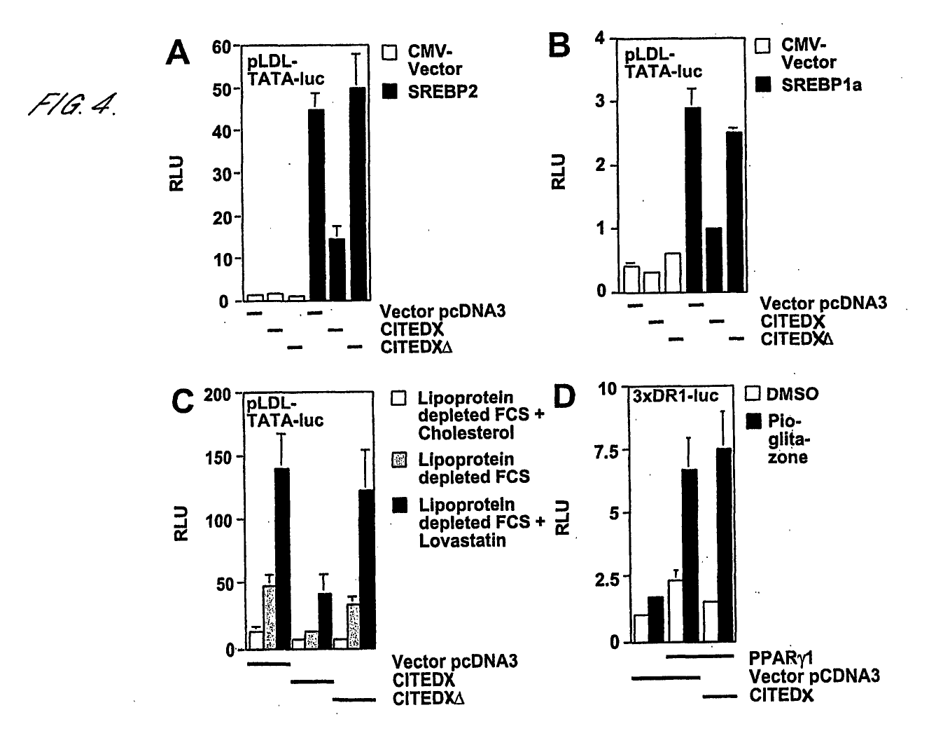

FIG. 4: HCITEDX inhibits SREBP but not PPARγ1 transactivation. (A) HCITEDX inhibits SREBP2 transactivation. Hep3B cells were co-transfected with 2 ng of SREBP2 expression vector, a reporter plasmid carrying the luciferase gene under the control of the LDL receptor promoter (pLDL-TATA-luc, 40 ng), CMV-lacZ (100 ng) and a plasmid expressing HCITEDX or HCITEDXΔ or the control pcDNA3 vector (150 ng). Results are presented as relative luciferase units (RLU), corrected for lacZ activity, and show the mean±SEM of three independent experiments. (B) HCITEDX inhibits SREBP1a transactivation. Hep3B cells were co-transfected as in (A), but with 40 ng of SREBP1a expression vector, and plasmids expressing HCITEDX, or HCITEDXΔ, or the control pcDNA3 vector (40 ng). Results are presented as for A. (C) HCITEDX inhibits transcription induced by cholesterol depletion. HepG2 cells were co-transfected with 40 ng of a plasmid carrying the luciferase gene under the control of the LDL promoter (pLDL-TATA-luc), CMV-lacZ (100 ng) and plasmids expressing HCITEDX or HCITEDXΔ or the control pcDNA3 vector (40 ng). Transfected cells were fed either with medium containing lipoprotein-depleted serum and 30 μM of lovastatin, lipoprotein-depleted serum only, or lipoprotein-depleted serum plus 10 μg/ml of cholesterol and 1 μg/ml of 25-hydroxycholesterol. Cells were cultured for an additional 18 hours before harvest. Results are presented as for A. (D) Effect of HCITEDX on PPARγ1 transactivation. Hep3B cells were co-transfected with expression vectors for mouse PPARγ1 (8 ng), 3×DR1-luc reporter (40 ng), CMV-lacZ (40 ng) and with either a plasmid expressing HCITEDX or the control pCDNA3 vector (40 ng). Cells were grown for 16 hours in the presence of DMSO (no ligand), or in the presence of 0.5 mM of pioglitazone dissolved in DMSO. Results are presented as for A.

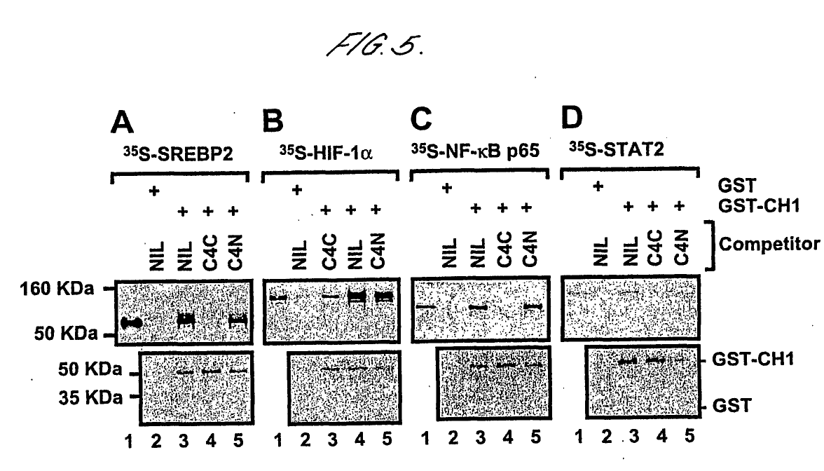

FIG. 5: HCITEDX blocks transcription factor interactions with p300-CH1.

(A-D) Top panels: Autoradiograms of SDS-PAGE gels. Lane 1 contains 20% of the input of the indicated transcription factor, which was generated as an in vitro translated ³⁵S-labelled peptide. The relative amount of the factor bound to bacterially expressed GST (lane 2), or to GST-p300CH1 (p300 residues 300-528, lanes 3-5), immobilized on glutathione-sepharose beads is shown. Binding was tested in the absence (lane 3), or in the presence of 20 mg of a synthetic peptide corresponding to the p300-CH1 binding domain of HCITEDX (lane 4, C4C), or in the presence of 20 mg of a control peptide from the N-terminus of mouse CITEDX (MCITEDX) (lane 5, C4N) Bottom panels: Coomassie stained gel showing relative amounts of GST and GST-p300CH1 proteins.

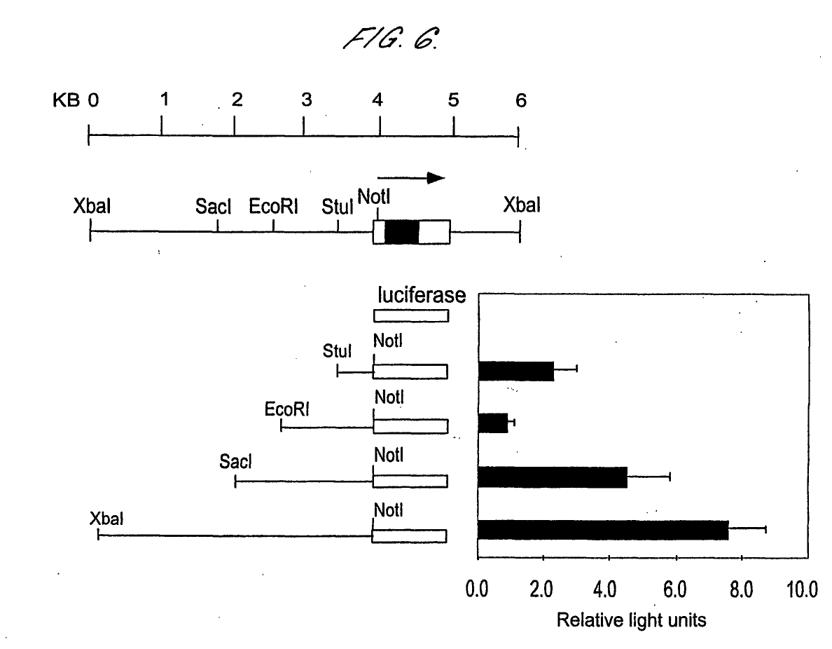

FIG. 6: The structure of the human CITEDX gene and 5′-flanking region is shown at the top. The open box indicates the single exon, the filled box the open reading frame. The arrow indicates the direction of transcription. Restriction enzyme sites are indicated (XbaI, SacI, EcoRI, StuI, NotI).

### MATERIAL AND METHODS

Standard molecular biology protocols which may be used in accordance with the invention may be found, for example, in: Sambrook et al. (1989), Molecular Cloning: A Laboratory Manual, Cold Spring Harbor Laboratory Press; F. M. Ausubel et al. (eds.), Current Protocols in Molecular Biology, John Wiley & Sons, Inc. (1994), plus regular updates.

Plasmids

VP16 and GAL4 mammalian two-hybrid plasmids were generated using pCMXGAL4N and pCMXVP16n (gifts from Ron Evans, Salk Institute, San Diego). GST-fusions were generated in pGEX4T1 (Pharmacia) following the instructions of the manufacturer. MBP fusions were generated in pMALC2 (NEB) following the instructions of the manufacturer.

The GAL4-SREBP2 (1-330), pcDNA-SREBP2 (1-462) LDL-TATA-luc and TATA-luc plasmids were gifts from Robert Tjian. The CMV-HSV-SREBP1a plasmid (full-length) was a gift from Joe Goldstein (University of Texas). GAL4-p300 fusions were gifts from Antonio Giordano (University of Pennsylvania). GAL4-SRC1 (789-883) was a gift from Tso-Pang Yao (Dana-Farber Cancer Institute, Boston Mass.). GAL4-EPSA1 (residues 19-870) was a gift from Chris Pugh (University of Oxford). GAL4-HIF-1a, GAL4-STAT2, GAL4-CH1, GAL4-CH1Δ and GST-p300-CH1 have been described previously (Bhattacharya et al., 1999). The IFN-α reporter plasmids have been described previously (Bhattacharya et al., 1996). CMV-NF_{κ}B-p65 and the 3X_{κ}B-INF-luc reporter were gifts from Xiaolu Yang and David Baltimore (MIT, Boston, Mass.). The GAL4-luc reporter and pCMX-lacZ plasmids were gifts from Ron Evans (Salk Institute, San Diego) and the VEGF-luc plasmid a gift from Mark Goldberg (Harvard Medical School, Boston). 3×DR1-luc reporter and pCMX-PPARγ1 were gifts from Bruce Spiegelman (Dana-Farber Cancer Institute, Boston, Mass.).

Antibodies

Polyclonal antibody to human HCITEDX was raised by immunising rabbits with GST-HCITEDX fusion protein. Anti-p300/CBP antibodies have been described previously (Eckner et al., 1996). PAB419 is a monoclonal antibody against SV40 Tag and this was a gift from Ed Harlow (MGH Cancer Centre, Boston Mass.). Antibody 9E10 is a gift from Jim DeCaprio.

Northern and Western Blotting.

These were performed using standard techniques (Ausubel et al. 1995). HCITEDX northern blots were probed with a 400 nt EcoNI fragment from IMAGE clone 1866039 Western blots for HCITEDX were transferred in Tris-glycine buffer with 20% methanol buffer onto PVDF membranes (Immobilon) which were blocked in 6% milk in PBS-T (PBS+0.1% Tween-20). Membranes were incubated with anti-HCITEDX serum (1:500) washed with PBS-T and probed with anti-rabbit IgG-HRP conjugate (1:200, Dako), or with Protein-A HRP (Amersham). Western blots for p300/CBP were performed using a combination of monoclonal antibodies AC26 and RW128 as previously described (Bhattacharya et al., 1999). Signals were detected by Supersignal (Pierce) following the instructions of the manufacturer.

GST Fusion—Binding and Peptide Competition.

GST fusion proteins were generated as described (Ausubel et al. 1995). ³⁵S labelled peptides were synthesised using a TNT kit (Promega) following the instructions of the manufacturer. In-vitro binding reactions were performed as previously described (Bhattacharya et al., 1999). Peptides used for competition experiments were DEEALTSLELELGLHRVRELPELFLGQSEFDCF (residues 138-170 of human CITEDX (HCITEDX)), and YAGPGMDSGLRPRGA (residues 32-46 of mouse CITEDX (MCITEDX)). GST-CH1 or GST, (1 μg of eluted protein) was pre-incubated with the indicated peptide (10 μg) for 5 hours in 300 Al Hyb-75+1% milk buffer. All reaction mixtures contained equal amounts of the DMSO vehicle used to dissolve the peptides. ³⁵S labelled in-vitro translated proteins were then added and binding reactions were carried out overnight, following which the relevant proteins were precipitated with glutathione-sepharose beads, and analysed as above.

Immunoprecipitations.

Immunoprecipitations were performed in buffer containing Tris (pH8) 50 mM, NaCl 150 mM, NP40 0.5%, EDTA 0.5 mM protease and phosphatase inhibitors (Roche complete protease inhibitor, PMSF 1 mM, sodium orthovanadate 0.5 mM, sodium fluoride 5 mM) and DTT 1 mM. Immunoprecipitations with AC240 anti-p300/CBP monoclonal antibody were performed essentially as described (Bhattacharya et al., 1999); 1.8 mg of ECV304 whole cell extract was used and the precipitates fractionated on a 5-20% SDS-PAGE gradient gel prior to western blotting. With the anti-HCITEDX antibody 1.2 mg of whole cell extract was used and the precipitates fractioned on a 5-12% step gradient SDS-PAGE gel.

Immunostaining.

HCITEDX immunostaining was performed as described (Eckner et al., 1994) with the anti-HCITEDX polyclonal antibody (1:250) and donkey anti-rabbit FITC secondary antibody (1:100, Chemicon). Nuclei were counterstained with TOPRO (Molecular Probes). Cells were visualised using confocal microscopy and data from blue and green channels were sequentially accumulated.

Cells.

Cells were cultivated in DMEM (Sigma) containing 10% fetal bovine serum (Sigma) supplemented with 10% fetal bovine scrum, 2 mM L-glutamine, 100 units/ml of penicillin, 100 μg/ml of streptomycin in a 6% CO2 containing atmosphere and passaged every 3 days. Hep3B and HepG2 cells were obtained from ATCC.

Transfections and Luciferase Assays.

Cells were plated in 24 well plates at 2.5×10⁴ cells per well, and were transfected the following day using Fugene 6 (Roche). Transfection mixtures contained 1 μl Fugene 6 and up to 0.5 μg of total plasmid per well, as recommended by the manufacturer. Cells were stimulated with hypoxia (1% oxygen), deferoxamine (100 μM, Sigma), pioglitazone (5 uM, gift from Pere Puigserver, Harvard Medicine School, Boston), TNF-α (10 ng/ml) or IFN-α2b (500 IU/ml), after 24 hours. Cells were harvested after 8 hours (for IFN-α2b) or 16 hours (for the other stimuli). Cholesterol-depletion experiments were performed as described (Mehta et al., 1996). Briefly the medium was replaced with DMEM containing 10% lipoprotein deficient serum (Sigma) after washing with serum free DEMEM. Lovastatin (30 μM, Calbiochem) or cholesterol (10 μg/ml Sigma) plus 25-hydroxycholesterol (1 μg/ml Sigma) was added as indicated and cells harvested after 18 hours. Luciferase and lacZ activities were measured and luciferase activity was corrected for lacZ activity to normalise for transfection efficiency and the data was presented as relative luciferase units (RLU). Amounts of DNA used per transfection refer to the amounts added per well of a 24 well plate.

### EXAMPLE 1

Cloning of Full Length HCITEDX

The 32 amino acid sequence encoding the p300-CH1 interacting domain of p35srj/CITED2 (DEEVLMSLVIEMGLDRIKELPELWLGQNEFDF) was used to search the GenBank expressed sequence tag (EST) database using the program TBLASTN. Several human, rat and mouse ESTs were identified.

Human EST clone 4568-m16 (IMAGE No. 1866039) and mouse EST clone 3138-j23 (IMAGE No. 1247350) were obtained from MRC-HGMP and the inserts were subcloned into pBluescript and other vectors. The human EST clone was used to screen a commercial human cDNA library according to standard procedures but no full length clones could be obtained using this approach. It was therefore necessary to devise an alternative and technically much more difficult approach based on the isolation of genomic clones in order to obtain a DNA clone containing the complete open reading frame of HCITEDX, as outlined below:

The insert from mouse EST clone 3138-j23 (IMAGE No. 1247350) was used to screen a mouse PAC genomic DNA library (129 Svev/TacfBr female mouse). Three independent positive clones were obtained. PAC DNA from the positive genomic clones was restriction mapped, Southern blotted and probed with fragments of a murine cDNA, the sequence of which was already present in the GenBank database (Accession No: AF143369). This cDNA, deposited in GenBank in April 1999, is the full length sequence of the murine homologue of HCITEDX, designated mrg2. Absence of rearrangements or internal deletions was indicated by the fact that all three clones had identical EcoRI and BamHI hybridising fragments, and that the EcoRI fragment co-migrated with the EcoRI fragment from 129 mouse genomic DNA. A 10.5 kb genomic EcoRI fragment that hybridised to both 5′ and 3′ murine Mrg2 cDNA sequences was subcloned into pBluescript and mapped using restriction enzymes and Southern blotting. Surprisingly, the restriction map distances indicated that the entire mouse mrg2 cDNA lay within-a single exon, a most unusual occurrence.

The inventors hypothesised that the human HCITEDX would share the same intron/exon structure as mrg2 and this was used as the basis for cloning the full length open reading frame of HCITEDX according to the following procedure. Standard molecular biology techniques were used. The insert from the human EST clone 4568-m16 (IMAGE No. 1866039) was used to screen a gridded human genomic PAC library (RPC121), and a positive clone was identified. The open reading frame of human HCITEDX was amplified from the human genomic clone template by PCR. The upstream primers used were either TS02.2:

5′-ggccgggatccaccATGGCCGACCACCTGATGCTCGCCGAGGGCTAC (with a Kozak sequence) or TS02:

5′-ggccgggatccGCCGACCACCTGATGCTCGCCGAGGGCTAC (without a Kozak or initial ATG, for generating N-terminal fusion proteins). The downstream primer was TS04:

5′ggccggtcgacTCAGCAGCTCACGGAGCCGGCGGG-CGGCG-CG-GACC.

The primer sequences were derived from the human EST sequences AI271898 and AI733502, which contain the 5′ and 3′ fragments of HCITEDX respectively. PCR products were digested with BamHI and SalI, and either subcloned into pcDNA3 (Invitrogen), or into pcDNA3-HA (where the HA epitope tag is inserted between the HindIII and BamHI sites). The inserts were sequenced on both strands, and sequences from independent PCR reactions were identical. SEQ ID NO: 2 shows the nucleotide sequence of the coding region of HCITEDX (552 nucleotides). The conceptual translation of this sequence is shown in SEQ ID NO: 1 (184 amino acids).

The HCITEDX gene was subsequently mapped to human chromosome 1p34.2-34.3 by fluorescent in-situ hybridisation using the genomic clone HPAC 409K1.

### EXAMPLE 2

Expression Patterns and Immunolocalisation

Northern hybridisation using an HCITEDX cDNA probe was carried out to determine the expression pattern of HCITEDX mRNA in various human tissues. A single HCITEDX transcript of 1.4 kb was detected in almost all human tissues tested (data not shown). HCITEDX transcript levels were highest in heart, liver, skeletal muscle and pancrease.

A polyclonal antibody against HCITEDX was raised by injecting rabbits with a glutathione S-transferase (GST) HCITEDX fusion protein. Endogenous HCITEDX was detected by western blotting in a number of cell lines including ECV304, a bladder tumour line with endothelial characteristics (data not shown). Endogenous HCITEDX protein migrates at approximately 24.5 kD.

The cellular localisation of endogenous HCITEDX in ECV304 cells was evaluated using immunostaining followed by confocal microscopy. The cellular levels of HCITEDX were observed to vary considerably. In some cells HCITEDX was undetectable, whereas in other it-was expressed predominantly in the nucleus, or both nucleus and cytoplasm (data not shown). In the absence of a specific nuclear localisation signal, the nuclear retention of HCITEDX, observed in at least some cells, suggests that it might be complexed at least some of the time with exclusively nuclear proteins. The variability in HCITEDX protein expression in individual cells suggests that it may be regulated through the cell-cycle.

### EXAMPLE 3

Binding of HCITEDX to R300 in vitro.

HCITEDX was shown to bind to the CH1 domain of p300 in an in vitro binding assay using ³⁵S labelled in vitro translated HCITEDX and GST tagged p300 (CH1 domain) immobilised on glutathione-agarose beads. Recombinant GST-p300 fusion proteins were produced in E. coli transformed with the vector pGEX-p300 (pGEX vectors are commercially available from Amersham Pharmacia Biotech, Uppsala, Sweden). Binding of HCITEDX to p300 was assessed by SDS-PAGE followed by autoradiography. Binding of HCITEDX to the CH1 domain of p300 in mammalian cells is also confirmed in the mammalian two-hybrid experiment, outlined below.

CITED2/p35srj interacts with CH1 domain of p300/CBP through a 32-amino acid sequence in its C-terminus (Bhattacharya et al., 1999). The C terminus of HCITEDX contains a homologous sequence, suggesting that it too should bind p300-CH1. To test this, plasmids were constructed expressing untagged, HA or VP16 transactivation domain-tagged full-length and carboxy-terminal mutant (lacking amino acids 138 to 184 and the putative CH1-binding domain) HCITEDX species. Vectors expressing human full-length CITED2/p35srj (as positive control), HCITEDX, or its truncated form were used to synthesise ³S-labelled proteins by coupled in vitro transcription-translation. These peptides were tested for interaction with GST-p300CH1 fusion protein expressed in bacteria (FIG. 2A). These experiments showed that, in vitro full-length HCITEDX specifically interacts with the CH1-domain of p300 as strongly as CITED2/p35srj. The carboxy-terminal region of HCITEDX extending between 138-184, is required for p300-CH1 binding.

### EXAMPLE 4

HCITEDX Inhibits Hypoxia-Activated Transcription

Hypoxia-induced factor-1α (HIF-1α) and its paralog HIF-2α/EPAS1, bind hypoxia-response elements in the promoters of target genes, recruit p300/CBP via the CH1 domain and activate transcription (Arany et al., 1996; Ema et al., 1999). We asked if HCITEDX affects HIF-1α and HIF-2α/EPAS1 transactivation. We used GAL4-HIF-1α, a fusion of HIF-1α residues 723-826 to the GAL4 DNA-binding domain. These HIF-1α residues bind p300-CH1 and constitute a transactivation domain. Co-transfection of GALA4-HIF-1α with a GAL4-luciferase reporter plasmid into cells activated the luciferase gene only after stimulation by hypoxia (FIG. 3A). Co-transfection of a HCITEDX expression plasmid (HA-HCITEDX) led to a complete loss of induced GAL4-HIF-1α transcriptional activity. A HCITEDX mutant lacking the p300-CH1 binding domain (HA-HCITEDXΔ) failed to interfere with GAL4-HIF-1α transcriptional activity. These results suggest that HCITEDX can interfere with the function of the HIF-1α C-terminal transactivation domain and that this effect requires the p300-CH1 binding domain of HCITEDX. In analogous experiments HCITEDX also inhibited the transactivation function of GAL4-EPAS1/HIF-2α (residues 19-870) (data not shown).

We also investigated whether HCITEDX inhibits the activation of natural HIF-1 response elements activated by hypoxia (FIG. 3B). A reporter plasmid containing the luciferase gene under the control of three HIF-1 consensus DNA-binding elements (3×HRE-luc), was used to assess the effect of HCITEDX expression on endogenous HIF-1 activity. As shown in FIG. 3B, transcription from the transfected reporter gene is activated when cells are stimulated by hypoxia and this is markedly inhibited by HCITEDX. The inhibition conferred by HCITEDX is dependent on the presence of its carboxy-terminal domain, since the expression of HCITEDXΔ is unable to suppress hypoxia-stimulated transcription. Taken togther, these results indicated that HCITEDX is an efficient inhibitor of hypoxia-induced transcription and of transactivation by HIF-1α and HIF-2α/EPAS1.

### EXAMPLE 5

HCITEDX Inhibits TNF-α Activated Transcription

The p65 subunit of NF-_{κ}B (NF-_{κ}B-p65) interacts with p300/CBP via the CH1 and the KIX domains (Hottiger et al., 1998; Zhong et al., 1998). NF-_{κ}B is activated by a large number of stimuli, including cytokines such as TNF-α. To investigate whether HCITEDX might affect transactivation by NF-_{κ}B, we performed transient transfection assays. A luciferase reporter plasmid containing three NF-_{κ}B-binding elements was transfected into Hep3B cells. This was strongly activated by co-transfecting a p65-NF-_{κ}B expression plasmid (FIG. 3C). Co-transfection of a HCITEDX expression plasmid led to a marked decrease of NF-_{κ}B transactivation. HCITEDX strongly inhibited TNF-α induced stimulation of NF-_{κ}B reporter activity (FIG. 3D), implying that it also inhibits transactivation by endogenous NF-_{κ}B. HCITEDXΔ had minimal effects, implying that efficient inhibition by HCITEDX requires a functional p300-CH1 binding domain.

### EXAMPLE 6

CITED Inhibits IFN-α Activated Transcription

STAT2 is activated by type I interferon (IFN-α or IFN-β), and transactivates target genes as part of the ISGF3 complex (reviewed in (Darnell, 1997) STAT2 interacts with p300 via the CH1 domain (Bhattacharya et al., 1996). To determine whether HCITEDX can affect STAT2 transactivation, we first tested its effect on a GAL4-STAT2 fusion protein, transactivation by which is dependent on its interaction with p300/CBP (Bhattacharya et al., 1996). As shown in FIG. 3E, GAL4-STAT2 efficiently activates a GAL4-luciferase reporter. Co-transfection of HA-HCITEDX led to a decrease of STAT2 transactivation, whereas no inhibitory effect was observed with HA-HCITEDXΔ. The effect of HA-HCITEDX is specific for STAT2, which interacts with the CH1 domain of P300/CBP, since it does not affect the activity of GAL4-SRC1, which interacts with the carboxy-terminus of p300/CBP (Yao et al., 1996). To confirm the physiological relevance of STAT2 inhibition by HCITEDX, we analysed its effects on a natural IFN-α responsive promoter, the IFN-α response element of the ISG54 gene (FIG. 3F). The reporter, when transfected into Hep3B cells, was efficiently activated by stimulation with IFN-α. Co-transfection of HCITEDX strongly inhibited IFN-α stimulated reporter activation. These results imply that HCITEDX inhibits STAT2-mediated transactivation in the context of the ISGF2 complex which is responsible for the activation of IFN-α responsive target genes. Again, HCITEDXΔ had minimal effect, implying that these effects of HCITEDX required a functional p300-CH1 binding domain.

### EXAMPLE 7

HCITEDX Inhibits Transcriptional Responses to Cholesterol Depletion

To further test whether HCITEDX affects the activity of transcription factors requiring the binding to p300/CBP at other domains than CH1, we tested its effect on transactivation by SREBP2 and SREBP1a. SREBPs mediate the transcriptional response to cholesterol depletion, and interact with the CREB-binding (KIX) domain of CBP (Oliner et al., 1996). We asked that HCITEDX would inhibit the activation of sterol response elements (SRE) by SREBP2. We performed co-transfection experiments in Hep3B cells, with expression vectors encoding SREBP2 OR SREBP1a, and a reporter plasmid containing the luciferase gene under the control of a single SRE in its native context within the LDL receptor promoter. Both SREBP2 (FIG. 4A) and SREBP1A (FIG. 4B) activated the transcription of the reporter plasmid. To our surprise HCITEDX markedly inhibited SREBP2 and SREBP1a transactivation. HCITEDXΔ had minimal effect in these experiments.

To determine if the inhibition of SREBP transactivation by HCITEDX occurred under natural reporter gene activation, we asked whether HCITEDX inhibits the activity of the LDL receptor promoter stimulated by cholesterol depletion. For this experiment, we used HepG2 cells, as Hep3B cells failed to efficiently activate the LDL receptor promoter transcription in response to sterol deprivation (data not shown). HepG2 cells were co-transfected with the LDL receptor promoter-luciferase reporter plasmid (FIG. 4C). The transcriptional activity of the promoter was suppressed when cholesterol and 25-hydroxycholesterol were added to the cell culture medium. The promoter was strongly activated when cells were maintained in medium lacking cholesterol (i.e. supplemented with lipoprotein depleted serum). Further induction was obtained by the addition of lovastatin, which blocks endogenous cholesterol biosynthesis, causing further cholesterol depletion (Goldstein and Brown, 1990). Co-transfection of a HCITEDX expression plasmid led to a marked decrease of reporter transactivation induced by cholesterol depletion. These results, taken together suggest that HCITEDX inhibits the transactivation function of SREBP1a and SREBP2, and blocks the transcriptional response to cholesterol deprivation. The lack of effect of HCITEDXΔ in these assays suggested that SREBP transactivation must require an interaction with p300/CBP-CH1.

### EXAMPLE 8

HCITEDX Effects are Specific to the D300/CBP-CH1 Domain

To test whether HCITEDX could interfere with transcription factors binding the amino-terminal domain of p300/CBP, we studied its effect on PPARγ1 (Gelman et al., 1999; Li et al., 2000). A plasmid harbouring the luciferase gene under the control of three PPAR-response elements was transfected into Hep3B cells (FIG. 4D). In cells stimulated with pioglitazone, a ligand that specifically activates PPARγ1 transactivation, the expression of luciferase was modestly activated, compared to the control cells. Co-transfection of a plasmid expressing PPARγ1, led to a substantial increase of the reporter plasmid activation during pioglitazone treatment. PPARγ1-dependent transcriptional activation was not affected by co-transfection of a HCITEDX expression plasmid. Similar results were obtained with PPARα, another transcription factor that binds the amino-terminus of P300/CBP (data not shown).

### EXAMPLE 9

CITED Inhibits Transcription Factor Interactions with p300-CH1

To determine the biochemical mechanism by which HCITEDX inhibited transactivation by SREBPs, we generated ³⁵S-labelled SREBP2 (residues 1-462) by coupled in vitro transcription-translation. This peptide was tested for interaction with bacterially expressed purified GST-CH1 fusion protein (FIG. 5A, lane 3). This experiment showed that, in vitro, SREBP2 interacts with the CH1-domain of p300. To determine if HCITEDX would affect its binding, we performed a similar in vitro binding experiment in the presence of a peptide corresponding to residues 138-170 of HCITEDX which is homologous to the p300CH1 binding domain previously mapped in CITED2 (Bhattacharya et al., 1999). Addition of the HCITEDX peptide in the reaction mix led to a complete loss of SREBP2 binding to the CH1 domain (FIG. 5A, lane 4). A control peptide derived from the N-terminus of HCITEDX had no effect (FIG. 5A, lane 5). Analogous experiments were performed using in vitro translated ³⁵S-labelled HIF-1α, NF-_{κ}B and STAT2 (FIG. 5B, C, and D). In each case, the HCITEDX peptide specifically inhibited the interaction of the relevant transcription factor with p300-CH1.

### EXAMPLE 10

Mammalian Two-Hybrid Assay

A mammalian two-hybrid assay was performed to determine the ability of various VP16-HCITEDX fusion proteins to interact GAL4-p300 full-length or truncated species in vivo (FIG. 2B and 2C). In these experiments, VP16-HCITEDX strongly and specifically activated a GAL4-luciferase reporter gene, when co-transfected with GAL4-p300CH1 indicating a two-hybrid interaction. No activation was observed with GAL4-CHG1Δ, which contains a mutant CH1 domain or with VP16-HCITEDXΔ which lacks HCITEDX residues 138-184 (FIG. 2C). As shown in FIG. 2C, a strong interaction between HCITEDX and p300 occurs only when the CH1 domain is preserved (GAL4-p300(1-596) and GAL4-p300(1-743), implying that the CH1 domain is necessary and sufficient for efficient binding of HCITEDX, HCITEDX also interacts with GAL4-p300 full-length, although this appears to be a weaker interaction. These results also confirm that the carboxy-terminal domain of HCITEDX is required for the interaction with CH1 domain and imply the absence of another p300 interaction domain in HCITEDX peptide.

Sources of plasmids used in these experiments are described under materials and methods. Combinations of plasmids were transiently co-transfected into Hep3B cells and luciferase activity was measured after 48 hours. Methods for transfection and measurement of luciferase activity were as described under materials and methods. In principle the experiment could be done in any mammalian cell line which is amenable to transfection.

In order to perform a compound screening assay the compound, at an appropriate dilution, is added to the transfected cells and luciferase activity is measured after an appropriate incubation.

### EXAMPLE 11

Endogenous HCITEDX—P300/CBP Complexes can be Recovered by Antibodies against P300/CBP and Against HCITEDX

To determine if this interaction occurred under physiological conditions, we investigated whether complexes between endogenous HCITEDX and P300/CBP proteins might be found in untransfected cells. Whole cell protein extracts from ECV304 cells were used for co-immunoprecipitation followed by western blotting experiments (FIG. 3D and 3E). Using a monoclonal anti-p300/CBP antibody (AC240), a fraction of endogenous HCITEDX is also co-immunoprecipitated and migrates just below the immunoglobulin light chain (FIG. 3D, middle panel, lane 3). Anti-HCITEDX antibody specifically immunoprecipitates the endogenous HCITEDX peptide (FIG. 3E, right, bottom panel, lane 2) and co-immunoprecipitates a fraction of the cellular p300/CBP (FIG. 3E, top panel, lane 2). P300/CBP proteins are not precipitated with the pre-immune serum (FIG. 3C, right, top panel, lane 3). These data indicated that, in vivo, at least a fraction of endogenous HCITEDX is associated with endogenous p300/CBP in stable, naturally occurring protein complexes.

### DISCUSSION OF EXAMPLES

Four major conclusions may be drawn from the Examples. First, HCITEDX, a new member of the CITED family, interacts physically with the transcriptional co-activators p300/CBP via the CH1 domain. Second, this interaction has significance for the function of this domain, as HCITEDX can inhibit transactivation by certain transcription factors that bind to p300/CBP-CH1. These factors include HIF-1α, HIF-2α/EPAS1, STAT2, NF-_{κ}B, and SREBPs. Third, HCITEDX competitively blocks the physical interaction between these proteins and p300-CH1. Fourth, HCITEDX strongly inhibits transcriptional activation by extra-cellular signals (hypoxia, type 1 interferon, TNF-α and sterol depletion) that depend on the above-mentioned transcription factors. These results suggest that one function of HCITEDX is to disrupt transcription factor interactions at p300/CBP-CH1, and also lend support to the idea that p300 and CBP are limiting in cells. They also imply a critical role for the interaction of these transcription factors with p300/CBP-CH1 and suggest that these interactions are potential pharmacological targets.

HCITEDX Interacts Physically with P300/CBP via the CH1 Domain:

Using specific monoclonal antibodies against p300/CBP, and polyclonal antibodies against HCITEDX we have shown that naturally occurring HCITEDX—P300/CBP complexes can be detected in untransfected cells. Using deletion mutants in binding assays in vitro and in mammalian 2-hybrid assays, we have defined the HCITEDX carboxy-terminus domain (residues 138-184) as the region required for the association with p300. In competition assays, we find that residues 138-170 are sufficient to inhibit transcription factor binding to p300-CH1. The CH1 domain of p300 is both necessary and sufficient for the interaction with HCITEDX and HCITEDX does not appear to interact with any other domain of p300.

HCITEDX Specifically Inhibits Transactivation by p330/CBP-CH1 Binding Transcription Factors:

Like other members of the CITED family and based on its interaction with P300/CBP, HCITEDX could function in at least two ways. First, it may recruit as yet undefined proteins to p300/CBP. Second, by binding P300/CBP-CH1, it may limit the available cellular pool of CH1 and inhibit transactivation by transcription factors that interact with CH1. Thus HCITEDX may function to modulate signal-activated transcriptional responses that depend on these factors.

Three dependent lines of evidence indicate that p300 and CBP are indeed functionally limiting in cells. First, a single wild-type of allele of p300 or CBP is unable to consistently promote wild-type function indicating that the genes are haploinsufficient (Kung et al., 2000a; Yao et al., 1998) (Petrij et al., 1995). Second, recruitment of p300/CBP by certain transcription factors such as nuclear hormone receptors and STAT1 inhibits the transcriptional function of other factors such as AP-1(Horvai et al., 1997; Kamei et al., 1996). Third, factors binding the CH1 domain of p300/CBP (e.g. CITED2/p35srj and HIF-1α, STAT2 and NF-_{κ}B) have been shown to compete for available CH1 sites (Bhattacharya et al., 1999; Hottiger et al., 1998).

Our data indicate that HCITEDX efficiently and specifically inhibits transactivation by certain transcription factors that are known to interact with p300/CBP-CH1. These factors include HIF-1α, HIF-2α/EPAS1, NF-_{κ}B and STAT2, and they play key roles, respectively, in the cellular responses to hypoxia, TNF-α and type I interferon. In keeping with this, we found that HCITEDX blocked the cellular transcriptional responses to hypoxia TNF-α and IFN-α. Surprisingly, and in contrast to our previous observations with CITED2/p35srj, we found that transactivation by SREBP2.was inhibited by HCITEDX.

The SREBP proteins play an essential role in the cellular transcriptional response to cholesterol depletion. Although they are known to bind p300/CBP, binding to the CH1 domain has never previously been demonstrated. Our data clearly indicate that SREBP2 directly binds p300-CH1 in vitro and that this interaction can be blocked by a 33-amino acid peptide sequence from HCITEDX a sequence that defines the CITED family. In keeping with this, we found that HCITEDX efficiently inhibits the cellular response to cholesterol depletion. It is not clear why HCITEDX functions more efficiently than CITED2/p35srj in this regard. One possibility is that CITED2/p35srj peptide is highly unstable, rendering it relatively less efficient.

The effects of HCITEDX appear to be specific to factors that bind p300/CBP-CH1. HCITEDX does not affect transactivation by the PPAR family of proteins which interact with the N-terminus of p300/CBP. Neither does it inhibit transactivation by SRC-1 which binds the carboxyl-terminus of p300/CBP. Thus HCITEDX is not a general inhibitor of cellular signalling pathways and its binding to p300/CBP-CH1 does not affect the general co-activation function of p300/CBP. Taken together, these results suggest that one function of HCITEDX may be to inhibit transcription factor interactions at p300/CBP-CH1 and also lend support to the idea that p300 and CBP are limiting in cells

A Critical Role for Transcription Factor Interactions at p300/CBP-CH1:

Transcription factors recruit co-activators such as P300-CBP by multiple protein interactions involving different domains. Moreover, they typically recruit multiple transcriptional co-activators. For instance HIF-1 (a heterodimer of HIF-1α and ARNT/HIF-1β) recruits p300/CBP via a HIF-1α-P300/CBP-CH1 interaction (Arany et al., 1996). In addition HIF-1 also recruits HNF-4 and SRC-1 which also links it to P300/CBP (Carrero et al., 2000; Huang et al., 1997). The interferon stimulated gene factor 3 (ISGF3) complex consisting of STAT2, STAT1, and p48 peptides recruits p300/CBP via an interaction between STAT2 and CH1 (Bhattacharya et al., 1996). STAT1 also binds the KIX and CH3 domains of P300/CBP (Zhang et al., 1996). The p65 sub-unit of NF-_{κ}B binds both KIX and CH1 domains of p300/CBP (Zhong et al., 1998). As shown in this study the SREBP proteins, which are known to bind the KIX domain (Oliner et al., 1996) also bind P300-CH1.

Our data suggest that the transactivation function of the above-mentioned transcription factors depend critically on their interaction with p300/CBP-CH1. This has implications for the pathophysiology and treatment of certain common human disease. HIF-1 plays an important role in tumour angiogenesis, and inhibition of HIF-1 function is likely to have anti-tumour effects (Kung et al., Ratcliffe et al., 2000). NF-_{κ}B plays a major role in inflammatory and immune responses, and NF-_{κ}B inhibition is likely to be useful in diseases where uncontrolled inflammatory or immune responses are part of the pathophysiology (reviewed in (Ghosh et al., 1998)). SREBP proteins transcriptionally up-regulate enzymes involved in cholesterol and lipid biosynthesis (reviewed in Brown and Goldstein, 1999)), and SREBP inhibitors may conceivably be useful in diseases such as atherosclerosis where hypercholesterolemia plays a major role. Our data implicate the p300/CBP-CH1 domain as a crucial component of different cellular pathways that play major roles in the pathophysiology of common diseases, and imply that these interactions are potential targets for pharmacological intervention.

### EXAMPLE 12

Analysis of the Human CITEDX Gene 5′-Flanking Region—it Functions as a Promoter

The structure of the human CITEDX gene and 5′-flanking region is shown at the top of FIG. 6. The open box indicates the single exon, the filled box the open reading frame. The arrow indicates the direction of transcription. Restriction enzyme sites are indicated (XbaI, SacI, EcoRI, StuI, NotI). The promoter activity of the human CITEDX 5′-flanking region was tested by fusing 4 kb (XbaI to NotI fragment, as shown in the figure), and deletions thereof (SacI-NotI, EcoRI to NotI, and StuI to NotI, as indicated in the figure) to a the luciferase reporter pGL3-basic (Promega), and transfecting the plasmids into U2-OS cells. The full-length construct (XbaI-NotI, 4 kb) was strongly (320 fold over basal activity) active in this assay. Deletions at the 5′-end of the insert resulted in a marked loss of activity, indicating that it contains cis-acting transcriptional activator sequences. Surprisingly, the luciferase activity was seen to increase in the StuI-NotI deletion construct. This implies that the EcoRI-StuI fragment likely contains a cis-acting repressor sequence.

Methods:

Transfection assays. Luciferase reporter constructs were transfected into human osteosarcoma (U2-OS) cells using Fugene 6 (Roche). U2-OS cells were maintained in DMEM supplemented with 10% FCS, penicillin streptomycin and L-glutamine. Cells were plated onto 24 well plates at a density of 2.5×10⁴ cells per well and grown overnight at 37° C. Each construct was transfected in duplicate. PCMV-lacZ (0.25 kg) was co-transfected with each test construct (0.1 μg) to correct for transfection efficiency. Luciferase and b-galactosidase activities were assayed 48 hours following transfection as described. Data (mean±SD, of 3 independent experiments) are presented as relative light units corrected for β-galactosidase activity.

This promoter may be used in pharmacological screens in transient or stably transfected cells to identify molecules that either up-regulate, or down-regulate HCITEDX production.

Sequence Listing

SEQ ID NO: 1 amino acid sequence of HCITEDX

SEQ ID NO: 2 nucleotide sequence of the HCITEDX open reading frame. The sequence shown is genomic DNA sequence, the coding region of HCITEDX being present in a single exon

SEQ ID NO: 3 genomic sequence for HCITEDX obtained by sequencing of a genomic clone

SEQ ID NO: 4 Primer TS02.2

SEQ ID NO:. 5 Primer (as TS02 but without Kozak sequence or initial ATG)

SEQ ID NO: 6 Primer TS04

SEQ ID NO: 7 Sequence of the XbaI-NotI fragment of the HCITEDX upstream region. This is in GenBank Human Genomic sequence AL158843.

References

Ghosh, S., M. J. May, and E. B. Kopp. 1998. NF-kappa B and Rel proteins: evolutionarily conserved mediators of immune responses. Annu Rev Immunol 16:225-60.

Andrews, J. E., M. J. O'Neill, M. Binder, T. Shioda, and A. H. Sinclair. 2000. Isolation and expression of a novel member of the CITED family. Mech Dev 95:305-8.

Ausubel, F., R. Brent, R. E. Kingston, D. D. Moore, J. G. Seidman, J. A. Smith, and K. Struhl. 1995. Short protocols in molecular biology, 3 ed. John Wiley & Sons, Inc.

Eckner, R., J. W. Ludlow, N. L. Lill, E. Oldread, Z. Arany, N. Modjtahedi, J. A. DeCaprio, D. M. Livingston, and J. A. Morgan. 1996. Association of p300 and CBP with simian virus 40 large T antigen. Mol Cell Biol 16:3454-64.

Ema, M., K. Hirota, J. Mimura, H. Abe, J. Yodoi, K. Sogawa, L. Poellinger, and Y. Fujii-Kuriyama. 1999. Molecular mechanisms of transcription activation by HLF and HIF1alpha in response to hypoxia: their stabilization and redox signal-induced interaction with CBP/p300. Embo J 18:1905-14.

Ericsson, J., and P. A. Edwards. 1998. CBP is required for sterol-regulated and sterol regulatory element-binding protein-regulated transcription. J Biol Chem 273:17865-70.

Fujita, T., M. Miyamoto, Y. Kimura, J. Hammer, and T. Taniguchi. 1989. Involvement of a cis-element that binds an H2TF-1/NF kappa B like factor(s) in the virus-induced interferon-beta gene expression. Nucleic Acids Res 17:3335-46.

Gelman, L., G. Zhou, L. Fajas, E. Raspe, J. C. Fruchart, and J. Auwerx. 1999. p300 interacts with the N- and C-terminal part of PPARgamma2 in a ligand-independent and -dependent manner, respectively. J Biol Chem 274:7681-8.

Glenn, D. J., and R. A. Maurer. 1999. MRG1 Binds to the LIM Domain of Lhx2 and May Function as a Coactivator to Stimulate Glycoprotein Hormone alpha-Subunit Gene Expression. J Biol Chem 274:36159-36167.

Goldstein, J. L., and M. S. Brown. 1990. Regulation of the mevalonate pathway. Nature 343:425-30.

Goodman, R. H., and S. Smolik. 2000. CBP/p300 in cell growth, transformation, and development. Genes Dev 14:1553-1577.

Grossman, S. R., M. Perez, A. L. Kung, M. Joseph, C. Mansur, Z. X. Xiao, S. Kumar, P. M. Howley, and D. M. Livingston. 1998. p300/MDM2 complexes participate in MDM2-mediated p53 degradation. Mol Cell 2:405-15.

Horvai, A. E., L. Xu, E. Korzus, G. Brard, D. Kalafus, T. M. Mullen, D. W. Rose, M. G. Rosenfeld, and C. K. Glass. 1997. Nuclear integration of JAK/STAT and Ras/AP-1 signaling by CBP and p300. Proc Natl Acad Sci USA 94:1074-9.

Hottiger, M. O., L. K. Felzien, and G. J. Nabel. 1998. Modulation of cytokine-induced HIV gene expression by competitive binding of transcription factors to the coactivator p300. Embo J 17:3124-34.

21. Huang, L. E., V. Ho, Z. Arany, D. Krainc, D. Galson, D. Tendler, D. M. Livingston, and H. F. Bunn. 1997. Erythropoietin gene regulation depends on heme-dependent oxygen sensing and assembly of interacting transcription factors. Kidney Int 51:548-52.

Kamei, Y., L. Xu, T. Heinzel, J. Torchia, R. Kurokawa, B. Gloss, S. C. Lin, R. A. Heyman, D. W. Rose, C. K. Glass, and M. G. Rosenfeld. 1996. A CBP integrator complex mediates transcriptional activation and AP-1 inhibition by nuclear receptors. Cell 85:403-14.

Kung, A. L., V. I. Rebel, R. T. Bronson, L. E. Ch'ng, C. A. Sieff, D. M. Livingston, and T. P. Yao. 2000. Gene dose-dependent control of hematopoiesis and hematologic tumor suppression by CBP. Genes Dev 14:272-7.

Kung, A. L., S. Wang, J. M. Klco, W. G. Kaelin, and D. M. Livingston. 2000. Suppression of tumor growth through disruption of hypoxia-inducible transcription. Nat Med 6:1335-1340.

Leung, M. K., T. Jones, C. L. Michels, D. M. Livingston, and S. Bhattacharya. 1999. Molecular cloning and chromosomal localization of the human CITED2 gene encoding p35srj/Mrg1. Genomics 61:307-13.

Li, M., G. Pascual, and C. K. Glass. 2000. Peroxisome proliferator-activated receptor gamma-dependent repression of the inducible nitric oxide synthase gene. Mol Cell Biol 20:4699-707.

Mehta, K. D., R. Chang, J. Underwood, J. Wise, and A. Kumar. 1996. Identification of a novel cis-acting element participating in maximal induction of the human low density lipoprotein receptor gene transcription in response to low cellular cholesterol levels. J Biol Chem 271:33616-22.

Oliner, J. D., J. M. Andresen, S. K. Hansen, S. Zhou, and R. Tjian. 1996. SREBP transcriptional activity is mediated through an interaction with the CREB-binding protein. Genes Dev 10:2903-11.

Perkins, N. D., L. K. Felzien, J. C. Betts, K. Leung, D. H. Beach, and G. J. Nabel. 1997. Regulation of NF-kappaB by cyclin-dependent kinases associated with the p300 coactivator. Science 275:523-7.

Petrij, F., R. H. Giles, H. G. Dauwerse, J. J. Saris, R. C. M. Hennekam, M. Masuno, N. Tommerup, G. B. Ommen, R. H. Goodman, D. J. M. Peters, and M. H. Breuning. 1995. Rubinstein-Taybi syndrome caused by mutations in the transcriptional co-activator CBP. Nature 376:348-351.

Ratcliffe, P. J., C. W. Pugh, and P. H. Maxwell. 2000. Targeting tumors through the HIF system. Nat Med 6:1315-1316.

Shikama, N., J. Lyon, and N. B. La Thangue. 1997. The p300/CBP family: integrating signals with transcription factors and chromatin. Trends Cell Biol 7:230-236.

Shioda, T., M. H. Fenner, and K. J. Isselbacher. 1996. msg1, a novel melanocyte-specific gene, encodes a nuclear protein and is associated with pigmentation. Proc Natl Acad Sci USA 93:12298-303.

Tian, H., S. L. McKnight, and D. W. Russell. 1997. Endothelial PAS domain protein 1 (EPAS1), a transcription factor selectively expressed in endothelial cells. Genes Dev 11:72-82.

Yahata, T., M. P. de Caestecker, R. J. Lechleider, S. Andriole, A. B. Roberts, K. J. Isselbacher, and T. Shioda. 2000. The MSG1

Petrij F, Giles RH, Dauwerse H G, et al. Rubinstein-Taybi syndrome caused by mutations in the transcriptional co-activator CBP. Nature 1995; 376: 348-351.

Pyeritz R E. Genetics and Cardiovascular disease. In: Braunwald E, ed. Heart Disease. Philadelphia: W B Saunders, 1997.

Shikama N, Lyon J, La Thangue N B. The p300/CBP family: integrating signals with transcription factors and chromatin. Trends Cell Biol 1997; 7:230-236.

Yao T P, Oh S P, Fuchs M, et al. Gene dosage-dependent embryonic development and proliferation defects in mice lacking the transcriptional integrator p300. Cell 1998; 93:361-72.

Bhattacharya S, Eckner R, Grossman S, Oldread E, Arany Z, D'Andrea A, Livingston D M. Cooperation of Stat2 and p300/CBP in signalling induced by interferon-alpha. Nature 1996; 383:344-7.

Paulson M, Pisharody S, Pan L, Guadagno S, Mui A L, Levy D E. Stat Protein Transactivation Domains Recruit p300/CBP through Widely Divergent Sequences. J Biol Chem 1999; 274:25343-25349.

Hottiger M O, Felzien L K, Nabel G J. Modulation of cytokine-induced HIV gene expression by competitive binding of transcription factors to the coactivator p300. Embo J 1998; 17:3124-34.

Arany Z, Huang L E, Eckner R, Bhattacharya S, Jiang C, Goldberg M A, Bunn H F, Livingston D M. An essential role for p300/CBP in the cellular response to hypoxia. Proc Natl Acad Sci USA 1996; 93:12969-73.

Kallio P J, Okamoto K, O'Brien S, Carrero P, Makino Y, Tanaka H, Poellinger L. Signal transduction in hypoxic cells: inducible nuclear translocation and recruitment of the CBP/p300 coactivator by the hypoxia-inducible factor-lalpha. Embo J 1998; 17:6573-86.

Ebert B L, Bunn H F. Regulation of transcription by hypoxia requires a multiprotein complex that includes hypozia-inducible factor 1, an adjacent transcription factor, and p300/CREB binding protein. Mol Cell Biol 1998; 18:4089-96.

Bhattacharya S, Michels C L, Leung M K, Arany Z P, Kung A L, Livingston D M. Functional role of p35srj, a novel p300/CBP binding protein, during transactivation by HIF-1. Genes Dev 1999; 13:64-75.

Ratclifee P, Rourke J, Maxwell P. Oxygen sensing, hypoxia-inducible factor-1 and the regulation of mammalian gene expression. J Exp Biol 1998; 201:1153-62.

Semenza G L, Agani F, Booth G, et al. Structural and functional analysis of hypoxia-inducible factor 1. Kidney Int 1997; 51:553-5.

Maxwell P H, Wiesener M S, Chang G W, et al. The tumour suppressor protein VHL targets hypoxia-inducible factors for oxygen-dependent proteolysis. Nature 1999; 399:271-5.

Dunwoodie S L, Rodriguez T A, Beddington R S P. Msg1 and Mrg1, founding members of a gene family, show distinct patterns of gene expression during mouse embryogenesis. Mech Dev 1998; 72:27-40.

1. An isolated nucleic acid molecule encoding an HCITEDX protein, said protein comprising the amino acid sequence illustrated in SEQ ID NO: 1.

2. A nucleic acid molecule according to claim 1 which comprises the complete nucleotide sequence illustrated in SEQ ID NO: 2 or SEQ ID NO: 3.

3. A nucleic acid molecule which is capable of hybridising to the nucleic acid molecule of claim 1 or claim 2 under conditions of high stringency.

4. A nucleic acid molecule encoding an HCITEDX protein, said protein comprising an amino acid sequence having at least 78% amino acid sequence identity with and a length approximately equal to the amino acid sequence illustrated in SEQ ID NO: 1.

5. A nucleic acid according to claim 3 which is an antisense molecule.

6. A nucleic acid molecule according to any one of claims 1 to 5 which is a DNA molecule.

7. An isolated HCITEDX protein comprising the amino acid sequence illustrated in SEQ ID NO: 1.

8. An isolated HCITEDX protein which is encoded by a nucleic acid molecule as defined in any one of claims 1 to 4.

9. A fragment of the HCITEDX protein of claim 7 or claim 8 which retains equivalent biological function.

10. An isolated nucleic acid molecule which comprises a sequence of nucleotides encoding a fragment of an HCITEDX protein as defined in claim 9.

11. An isolated nucleic acid molecule which comprises the complete nucleotide sequence illustrated in SEQ ID NO: 2 or the complement thereof.

12. A nucleic acid molecule according to claim 11 which comprises the complete nucleotide sequence illustrated in SEQ ID NO: 3.

13. An isolated nucleic acid molecule according to claim 11 or claim 12 which is DNA, preferably genomic DNA or cDNA, RNA or PNA.

14. An expression vector comprising a sequence of nucleotides which encodes an HCITEDX protein, said protein comprising the amino acid sequence illustrated in SEQ ID NO: 1.

15. An expression vector according to claim 14 wherein the sequence of nucleotides encoding the HCITEDX protein comprises the complete nucleotide sequence illustrated in SEQ ID NO: 2 or SEQ ID NO: 3.

16. An expression vector comprising a sequence of nucleotides which encodes a fragment of an HCITEDX protein as defined in claim 9.

17. An expression vector according to any one of claims 14 to 16 which is an adenoviral vector.

18. An expression vector which is adapted for the expression of a fusion protein, the fusion protein comprising an HCITEDX protein as defined claim 7 or claim 8 or a fragment thereof as defined in claim 9 fused in-frame to at least one heterologous protein or polypeptide.

19. A host cell comprising the expression vector of any one of claims 14 to 19.

20. A non-human transgenic organism comprising a transgene capable of expressing an HCITEDX protein according to claim 7 or claim 8 or a fragment thereof as defined in claim 9.

21. A transgenic organism according to claim 20 wherein the transgene comprises the complete nucleotide sequence illustrated in SEQ ID NO: 2 or SEQ ID NO: 3.

22. An oligonucleotide molecule comprising a sequence of between about 10 and 50 consecutive nucleotides from the nucleotide sequence illustrated in SEQ ID NO: 2 or the complement thereof.

23. An antibody directed to an epitope of the HCITEDX protein claimed in claim 7 or claim 8.

24. A protein composition comprising the HCITEDX protein of claim 7 or claim 8 linked to a protein transduction domain peptide which is capable of mediating protein transduction into mammalian cells in vivo.

25. A protein composition according to claim 24 wherein the HCITEDX molecule is fused N-terminally to the protein transduction domain peptide.

26. A protein composition according to claim 43 or claim 44 wherein the protein transduction domain peptide is the protein transduction domain from the human immunodeficiency virus TAT protein or a synthetic variant thereof having equivalent function.

27. A medicament comprising an HCITEDX protein according to claim 7 or claim 8 or a protein composition as defined in any one of claims 44 to 45 and a pharmaceutically acceptable carrier, diluent or excipient.

28. A medicament including as the pharmaceutically active ingredient a polypeptide comprising at least amino acids 138 to 170 of SEQ ID NO: 1.

29. A method for identifying compounds which modulate the interaction between HCITEDX and the CH1 domain of p300/CBP, which method comprises:
providing a host cell containing a DNA construct comprising a reporter gene or a counter-selectable marker gene operably linked to a promoter regulated by a transcription factor having a DNA binding domain and an activating domain;
expressing in said host cell a first hybrid DNA sequence encoding a first hybrid protein comprising the HCITEDX protein of claim 7 or claim 8 or a fragment thereof including the p300 binding domain fused in-frame to either the DNA binding domain or the activating domain of the said transcription factor;
expressing in said host cell a second hybrid DNA sequence encoding a second hybrid protein comprising p300 or a fragment thereof including the CH1 domain fused in-frame to either the DNA binding domain or the activating domain of the said transcription factor, such that when the first fusion protein comprises the activation domain of the said transcription factor the second fusion protein comprises the DNA binding domain of the said transcription factor and when the first fusion protein comprises the DNA binding domain of the transcription factor the second fusion protein comprises the activation domain;
contacting the host cell with a sample of a candidate compound; and
detecting any binding of the HCITEDX protein or fragment thereof to the p300 CH1 domain by either detecting the production of any reporter gene product in the said host cell or by applying positive selection for loss of expression of the counter-selectable marker gene.

30. A method according to claim 29 wherein the first fusion protein comprises the HCITEDX protein of claim 7 or claim 8 or a fragment thereof including the p300 binding domain fused in-frame to the activation domain of VP16, the second fusion protein comprises p300 or a fragment thereof including the CH1 domain fused in-frame to the DNA binding domain of GAL4 and the host cell contains a DNA construct comprises a luciferase gene operably linked to the GAL4 promoter.

31. A method according to claim 29 or claim 30 wherein the host cell is a mammalian cell.

32. A method according to claim 31 wherein the host cell is an hepatocyte or an immortalised cell derived from an hepatocyte.

33. An in vitro method for identifying compounds which modulate the interaction between HCITEDX and the CH1 domain of p300/CBP, which method comprises:
forming a mixture comprising a first protein component comprising an HCITEDX protein according to claim 7 or claim 8 or a fragment thereof including the p300 binding domain, a second protein component comprising p300 protein or a fragment thereof including the CH1 domain, and a candidate compound,
incubating the mixture under conditions which, in the absence of the candidate compound, would permit binding of the HCITEDX protein to the CH1 domain of p300, and
detecting any binding of the HCITEDX protein to the CH1 domain of p300.

34. A compound which is identifiable as a modulator of the interaction between HCITEDX and the CH1 domain of p300/CBP using the method of any one of claims 29 to 33.

35. An in vitro method for identifying compounds which modulate the cytoplasmic sequestration of the HCITEDX protein, which method comprises:
contacting a mammalian cell expressing an HCITEDX protein according to claim 7 or claim 8 with a candidate compound and detecting any changes in the cellular localisation of the said HCITEDX protein in the presence of the compound.

36. A method according to claim 35 wherein the HCITEDX protein is labelled.

37. A method according to claim 36 wherein the label is genetically encoded.

38. A method according to claim 37 wherein the genetically encoded label is an epitope tag and changes in the cellular localisation of the HCITEDX protein are detected by immunofluorescence using an antibody specific for the epitope tag.

39. A method according to claim 38 wherein the genetically encoded label is an autonomously fluorescent protein.

40. A method according to any one of claims 35 to 39 wherein the mammalian cell is an Hep3B or U2OS.

41. A compound which is identifiable as modulating the cytoplasmic sequestration of the HCITEDX protein using the method of any one of claims 34 to 40.

42. An in vitro method for identifying compounds which modulate the interaction between SREBP1 or SREBP2 and the CH1 domain of p300/CBP, which method comprises:
forming a mixture comprising a first protein component comprising either SREBP1 or SREBP2, a second protein component comprising the CH1 domain of p300, and a candidate compound,
incubating the mixture under conditions which, in the absence of the candidate compound, would permit binding of SREBP to the CH1 domain of p300, and
detecting any binding of either SREBP1 or SREBP2 to the CH1 domain of p300.

43. A method for identifying compounds which modulate the interaction between SREBP1 or SREBP2 and the CH1 domain of p300/CBP, which method comprises:
providing a host cell containing a DNA construct comprising a reporter gene or a counter-selectable marker gene operably linked to a promoter regulated by a transcription factor having a DNA binding domain and an activating domain;
expressing in said host cell a first hybrid DNA sequence encoding a first hybrid protein comprising either SREBP1 or SREBP2 fused in-frame to either the DNA binding domain or the activating domain of the said transcription factor;
expressing in said host cell a second hybrid DNA sequence encoding a second hybrid protein comprising the CH1 domain of p300 fused in-frame to either the DNA binding domain or the activating domain of the said transcription factor, such that when the first fusion protein comprises the activation domain of the said transcription factor the second fusion protein comprises the DNA binding domain of the said transcription factor and when the first fusion protein comprises the DNA binding domain of the transcription factor the second fusion protein comprises the activation domain;
contacting the host cell with a sample of a candidate compound; and
detecting any binding of either SREBP2 or SREBP1 to the p300 CH1 domain by either detecting the production of any reporter gene product in the said host cell or by applying positive selection for loss of expression of the counter-selectable marker gene.

44. A compound which is identifiable as a modulator of the interaction between SREBP1 and/or SREBP2 and the CH1 domain of p300/CBP using the method of claim 42 or claim 43.

45. An in vitro method for identifying compounds which modulate the interaction between NF-κB-65 and the CH1 domain of p300/CBP, which method comprises:
forming a mixture comprising a first protein component comprising NF-κB-p65, a second protein component comprising the CH1 domain of p300, and a candidate compound,
incubating the mixture under conditions which, in the absence of the candidate compound, would permit binding of NF-κB-p65 to the CH1 domain of p300, and
detecting any binding of NF-κB-p65 to the CH1 domain of p300.

46. A method for identifying compounds which modulate-the interaction between NF-κB-p65 and the CH1 domain of p300/CBP, which method comprises:
providing a host cell containing a DNA construct comprising a reporter gene or a counter-selectable marker gene operably linked to a promoter regulated by a transcription factor having a DNA binding domain and an activating domain;
expressing in said host cell a first hybrid DNA sequence encoding a first hybrid protein comprising NF-κB-p65 fused in-frame to either the DNA binding domain or the activating domain of the said transcription factor;
expressing in said host cell a second hybrid DNA sequence encoding a second hybrid protein comprising p300 or a fragment thereof including the CH1 domain fused in-frame to either the DNA binding domain or the activating domain of the said transcription factor, such that when the first fusion protein comprises the activation domain of the said transcription factor the second fusion protein comprises the DNA binding domain of the said transcription factor and when the first fusion protein comprises the DNA binding domain of the transcription factor the second fusion protein comprises the activation domain;
contacting the host cell with a sample of a candidate compound; and
detecting any binding of NF-κB-p65 to the p300 CH1 domain by either detecting the production of any reporter gene product in the said host cell or by applying positive selection for loss of expression of the counter-selectable marker gene.

47. A compound which is identifiable as a modulator of the interaction between NF-κB-p65 and the CH1 domain of p300/CBP using the method of claim 45 or claim 46.

48. An isolated HCITEDX promoter fragment having the sequence of nucleotides illustrated in SEQ ID NO: 7.

49. A method of identifying a compound capable of modulating expression of HCITEDX from its natural promoter, which method comprises:
providing a recombinant host cell containing a reporter gene expression construct comprising the promoter region of the human HCITEDX gene operably linked to a reporter gene;
contacting the host cell with a candidate compound; and
screening for expression of the reporter gene product.
